(19) United States
(12) Patent Application Publication
(10) Pub. No.: US 2007/0276019 A1
(43) Pub. Date: Nov. 29, 2007

(54) Haloalkyl Containing Compounds as Cysteine Protease Inhibitors

(75) Inventor: John O. Link, San Francisco, CA (US)

Correspondence Address: MILLEN, WHITE, ZELANO AND BRANIGAN, P.C.;C/O BERLEX BIOSCIENCES, 2200 CLARENDON BOUEVARD, SUITE 1400, ARLINGTON, VA, 22201, US

(73) Assignee: Axys Pharmaceuticals, Inc., So. San Francisco, CA (US)

(21) Appl. No.: 10/570,885

(22) PCT Filed: Sep. 17, 2004
(86) PCT No.: PCT/US04/30438

§ 371 Date: Dec. 15, 2006

Related U.S. Application Data
(60) Provisional application No. 60/504,167, filed on Sep. 18, 2003.

(51) Int. Cl. C07D 263/56; A61K 31/16; A61K 31/423; A61P 17/06; A61P 37/00; C07C 233/02

(52) U.S. Cl. 514/375; 514/613; 548/217; 564/193

(57) ABSTRACT

The present invention is directed to compounds that are inhibitors of cysteine proteases, in particular, cathepsins B, K, L, F, and S and are therefore useful in treating diseases mediated by these proteases. The present invention is directed to pharmaceutical compositions comprising these compounds and processes for preparing them.

### FIELD OF THE INVENTION

The present invention is directed to compounds that are inhibitors of cysteine proteases, in particular, cathepsins B, K, L, F, and S and are therefore useful in treating diseases mediated by these proteases. The present invention is directed to pharmaceutical compositions comprising these compounds and processes for preparing them.

### STATE OF THE ART

Cysteine proteases represent a class of peptidases characterized by the presence of a cysteine residue in the catalytic site of the enzyme. Cysteine proteases are associated with the normal degradation and processing of proteins. The aberrant activity of cysteine proteases, e.g., as a result of increased expression or enhanced activation, however, may have pathological consequences. In this regard, certain cysteine proteases are associated with a number of disease states, including arthritis, muscular dystrophy, inflammation, tumor invasion, glomerulonephritis, malaria, periodontal disease, metachromatic leukodystrophy and others. For example, increased cathepsin B levels and redistribution of the enzyme are found in tumors; thus, suggesting a role for the enzyme in tumor invasion and metastasis. In addition, aberrant cathepsin B activity is implicated in such disease states as rheumatoid arthritis, osteoarthritis, pneumocystis carinii, acute pancreatitis, inflammatory airway disease and bone and joint disorders.

The prominent expression of cathepsin K in osteoclasts and osteoclast-related multinucleated cells and its high collagenolytic activity suggest that the enzyme is involved in ososteoclast-mediated bone resorption and, hence, in bone abnormalities such as occurs in osteoporosis. In addition, cathepsin K expression in the lung and its elastinolytic activity suggest that the enzyme plays a role in pulmonary disorders as well.

Cathepsin L is implicated in normal lysosomal proteolysis as well as several disease states, including, but not limited to, metastasis of melanomas. Cathepsin S is implicated in Alzheimer's disease and certain autoimmune disorders, including, but not limited to juvenile onset diabetes, multiple sclerosis, pemphigus vulgaris, Graves' disease, myasthenia gravis, systemic lupus erythemotasus, rheumatoid arthritis and Hashimoto's thyroiditis. In addition, cathepsin S is implicated in: allergic disorders, including, but not limited to asthma; and allogeneic immune responses, including, but not limited to, rejection of organ transplants or tissue grafts.

In view of the number of diseases wherein it is recognized that an increase in cysteine protease activity contributes to the pathology and/or symptomatology of the disease, molecules which inhibit the activity of this class of enzymes, in particular molecules which inhibitor cathepsins B, K, L, F, and/or S, will therefore be useful as therapeutic agents.

### SUMMARY OF THE INVENTION

In one aspect, this invention is directed to a compound of Formula (I): 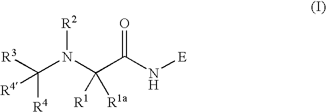
wherein:

E is:

(i) —C(R⁵)(R⁶)X¹ where X¹ is —CHO, —C(R⁷)(R⁸)CF₃, —C(R⁷)(R⁸)CF₂CF₂R⁹, —C(R⁷)(R⁸)R¹⁰, —CH═CHS(O)₂R¹⁰, —C(R⁷)(R⁸)C(R⁷)(R⁸)OR¹⁰, —C(R⁷)(R⁸)CH₂OR¹⁰, —C(R⁷)(R⁸)C(R⁷)(R⁸)R¹⁰, —C(R⁷)(R⁸)CH₂N(R¹¹)SO₂R¹⁰, —C(R⁷)(R⁸)CF₂C(O)NR¹⁰R¹¹, —C(R⁷)(R⁸)C(O)NR¹⁰R¹¹, —C(R⁷)(R⁸)C(O)N(R¹¹)(CH₂)₂OR¹¹, or —C(R⁷)(R⁸)C(O)N(R¹¹)(CH₂)₂NR¹⁰R¹¹ where:

R⁵ is hydrogen or alkyl; and

R⁶ is selected from the group consisting of hydrogen, alkyl, haloalkyl, carboxyalkyl, alkoxycarbonylalkyl, cycloalkyl, cycloalkylalkyl, aryl, aralkyl, heteroaryl, heteroaralkyl, heterocyclyl, heterocyclylalkyl, cyano, -alkylene-X²—R¹² (where X² is —O—, —NR¹³—, —CONR¹³—, —S(O)ₙ₁—, —NHCO—, —CO—, or —C(O)O— where n1 is 0-2, and R¹² and R¹³ are independently hydrogen, alkyl, haloalkyl, cycloalkyl, cycloalkylalkyl, aryl, aralkyl, heteroaryl, or heteroaralkyl) wherein the aromatic or alicyclic ring in R⁶ is optionally substituted with one, two, or three R^{a} independently selected from alkyl, haloalkyl, alkoxy, hydroxy, haloalkoxy, halo, carboxy, alkoxycarbonyl, amino, monosubstituted amino, disubstituted amino, nitro, aryloxy, benzyloxy, acyl, or arylsulfonyl and further where the aromatic or alicyclic ring in R^{a} is optionally substituted with one or two substituents independently selected from alkyl, halo, alkoxy, haloalkyl, haloalkoxy, hydroxy, amino, alkylamino, dialkylamino, carboxy, or alkoxycarbonyl; or

R⁵ and R⁶ taken together with the carbon atom to which both R⁵ and R⁶ are attached form (i) cycloalkylene optionally substituted with one or two R^{b} independently selected from alkyl, halo, alkylamino, dialkylamino, aryl, aralkyl, cycloalkyl, cycloalkylalkyl, heteroaryl, heteroaralkyl, alkoxycarbonyl, or aryloxycarbonyl, or (ii) heterocyclylalkylene optionally substituted with one to four R^{c} which are independently selected from alkyl, haloalkyl, hydroxy, hydroxyalkyl, alkoxyalkyl, alkoxyalkyloxyalkyl, aryloxyalkyl, heteroaryloxyalkyl, aminoalkyl, acyl, aryl, aralkyl, heteroaryl, heteroaralkyl, heterocyclyl, heterocyclylalkyl, cycloalkyl, cycloalkylalkyl, —S(O)ₙ₂R¹⁴, alkylene-S(O)ₙ₂—R¹⁵, —COOR⁶, -alkylene-COOR¹⁷, —CONHR¹⁸R¹⁹, or -alkylene-CONHR²⁰R²¹ (where n2 is 0-2 and R₁₄-R¹⁷, R¹⁸ and R²⁰ are independently hydrogen, alkyl, haloalkyl, aryl, aralkyl, heteroaryl, heteroaralkyl, cycloalkyl, cycloalkylalkyl, or heterocyclyl and R¹⁹ and R²¹ are independently hydrogen or alkyl) and further wherein the aromatic or alicyclic ring in the groups attached to cycloalkylene or heterocyclylalkylene is optionally substituted with one, two, or three substituents independently selected from alkyl, haloalkyl, alkoxy, hydroxy, haloalkoxy, halo, carboxy, alkoxycarbonyl, amino, monosubstituted amino, disubstituted amino, or acyl;

R⁷ is hydrogen or alkyl;

R⁸ is hydroxy; or

R⁷ and R⁸ together form oxo;

R⁹ is hydrogen, halo, alkyl, aralkyl or heteroaralkyl;

R¹⁰ is hydrogen, alkyl, aryl, aralkyl, heteroaryl, heteroaralkyl, cycloalkyl, cycloalkylalkyl, heterocyclyl, or heterocyclylalkyl wherein the aromatic or alicyclic ring in R¹⁰ is optionally substituted with one, two, or three R^{d} independently selected from alkyl, haloalkyl, alkoxy, cycloalkyl, hydroxy, haloalkoxy, halo, carboxy, alkoxycarbonyl, aryl, heteroaryl, amino, monosubstituted amino, disubstituted amino, or acyl and further wherein the aromatic or alicyclic ring in R^{d} is optionally substituted with one, two, or three substitutents independently selected from alkyl, haloalkyl, alkoxy, haloalkoxy, halo, hydroxy, carboxy, alkoxycarbonyl, amino, alkylamino, or dialkylamino; and

R¹¹ is hydrogen or alkyl; or

(ii) a group of formula (a): 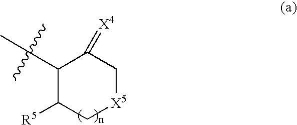
where:

n is 0, 1, or 2;

X⁴ is selected from —NR²²—, —S—, or —O— where R²² is hydrogen, alkyl, or alkoxy; and

X⁵ is —O—, —S—, —SO₂—, or —NR²³— where R²³ is selected from hydrogen, alkyl, haloalkyl, hydroxyalkyl, alkoxyalkyl, aryloxyalkyl, heteroaryloxyalkyl, aminoalkyl, acyl, aryl, aralkyl, heteroaryl, heteroaralkyl, cycloalkyl, cycloalkylalkyl, —S(O)₂R²⁴, -alkylene-S(O)ₙ₃—R²⁵, —COOR²⁶, -alkylene-COOR²⁷, —CONR²⁸R²⁹, or -alkylene-CONR³⁰R³¹ (where n3 is 0-2, R²⁴-R²⁷, R²⁸ and R³⁰ are independently hydrogen, alkyl, haloalkyl, aryl, aralkyl, heteroaryl, heteroaralkyl, cycloalkyl, cycloalkylalkyl, heterocyclyl, or heterocyclylalkyl, and R²⁹ and R³¹ are independently hydrogen or alkyl) where the aromatic or alicyclic ring in the groups attached to X⁵ is optionally substituted with one, two, or three substituents independently selected from alkyl, haloalkyl, alkoxy, haloalkoxy, halo, hydroxy, amino, alkylamino, dialkylamino, carboxy, or alkoxycarbonyl; and

R⁵ is as defined above;

R¹ is hydrogen or alkyl;

R^{1a} is hydrogen, alkyl, haloalkyl, cycloalkyl, cycloalkylalkyl, aryl, aralkyl, heteroaryl, heteroaralkyl, heterocyclylalkyl, or -alkylene-X⁶—R³² (wherein X⁶ is —NR³³—, —O—, —S(O)ₙ₄—, —CO—, —COO—, —OCO—, —NR³³CO—, —CONR³³—, —NR³³SO₂—, —SO₂NR³³—, —NR³³COO—, —OCONR³³—, —NR³³CONR³⁴, or —NR³³SO₂NR³⁴— where R³³ and R³⁴ are independently hydrogen, alkyl, or acyl, n4 is 0-2, and R³² is hydrogen, alkyl, haloalkyl, cycloalkyl, cycloalkylalkyl, aryl, aralkyl, heteroaryl, heteroaralkyl, heterocyclyl, or heterocyclylalkyl) wherein said alkylene chain in -alkylene-X⁶—R³² is optionally substituted with one to six halo and wherein the aromatic or alicyclic ring in R^{1a} is optionally substituted with one, two, or three R^{e} independently selected from alkyl, haloalkyl, alkoxy, hydroxy, haloalkoxy, halo, nitro, cyano, carboxy, alkoxycarbonyl, aryl, heteroaryl, cycloalkyl, cycloalkylalkyl, aralkyl, heteroaralkyl, heterocyclyl, amino, monosubstituted amino, disubstituted amino, acyl, or -(alkylene)ₘ—X⁷—R³⁵ (wherein X⁷ is —NR³⁶—, —O—, —S(O)ₙ₅—, —CO—, —COO—, —OCO—, —NR³⁶CO—, —CONR³⁶—, —NR³⁶SO₂—, —SO₂NR³⁶—, —NR³⁶COO—, —OCONR³⁶—, —NR³⁶CONR³⁷—, or —NR³⁶SO₂NR³⁷— where R³⁶ and R³⁷ are independently hydrogen, alkyl, or acyl and m is 0 or 1, and n5 is 0-2, and R³⁵ is cycloalkyl, cycloalkylalkyl, aryl, aralkyl, heteroaryl, heteroaralkyl, heterocylyl or heterocyclylalkyl) wherein the aromatic or alicyclic ring in R^{e} is optionally substituted with one, two, or three substituents independently selected from alkyl, alkoxy, alkoxyalkyl, alkylsulfonyl, alkylsulfonylalkyl, alkylaminosulfonyl, acyl, halo, haloalkyl, haloalkoxy, cyano, nitro, hydroxy, hydroxyalkyl, carboxy, alkoxycarbonyl, aryl optionally substituted with alkoxy or halo, aralkyl optionally substituted with alkoxy or halo, aryloxy optionally substituted with alkoxy or halo, heteroaryl optionally substituted with alkoxy or halo, or heteroaralkyl optionally substituted with alkoxy or halo, amino, aminosulfonyl, alkylamino, dialkylamino, or alkynyl optionally substituted with hydroxy, aryl, or heteroaryl; or

R¹ and R^{1a} together with the carbon atoms to which they are attached form cycloalkylene or heterocyclylalkylene ring wherein said cycloalkylene or heterocyclylalkylene is optionally substituted with one or two R^{f} independently selected from alkyl, halo, hydroxyalkyl, keto, or —SO₂R where R is alkyl, cycloalkyl, cycloalkylalkyl, aryl, aralkyl, heteroaryl or heteroaralkyl and further where the aromatic or alicylic ring in R^{f} is optionally substituted with one, two, or three substitutents independently selected from alkyl, alkoxy, haloalkyl, haloalkoxy, hydroxy, halo, carboxy, or alkoxycarbonyl;

R² is hydrogen or alkyl;

R³ is hydrogen, alkyl, haloalkyl, cycloalkyl, aryl, aralkyl, heteroaryl, heteroaralkyl, heterocyclyl, heterocyclylalkyl, or -alkylene-X⁸—R³⁸ (wherein X⁸ is —NR³⁹—, —O—, —S(O)ₙ₆—, —CO—, —COO—, —OCO—, —NR³⁹CO—, —CONR³⁹—, —NR³⁹SO₂—, —SO₂NR³⁹—, —NR³⁹COO—, —OCONR³⁹—, —NR³⁹CONR⁴⁰—, or —NR³⁹SO₂NR⁴⁰— where R³⁹ and R⁴⁰ are independently hydrogen, alkyl, or acyl, n6 is 0-2, and R³⁸ is hydrogen, alkyl, haloalkyl, cycloalkyl, cycloalkylalkyl, aryl, aralkyl, heteroaryl, or heteroaralkyl) wherein the aromatic or alicyclic rings in R³ are optionally substituted with one, two, or three R^{g} independently selected from alkyl, halo, hydroxy, alkoxy, haloalkyl, haloalkoxy, oxo, cyano, nitro, acyl, acyloxy, aryl, aralkyl, heteroaryl, heteroaralkyl, cycloalkyl, cycloalkylalkyl, heterocyclyl, heterocyclylalkyl, aryloxy, benzyloxy, carboxy, alkoxycarbonyl, aryloxycarbonyl, carbamoyl, alkylthio, alkylsulfinyl, alkylsulfonyl, arylthio, arylsulfonyl, arylsulfinyl, alkoxycarbonylamino, aryloxycarbonylamino, alkylcarbamoyloxy, arylcarbamoyloxy, alkylsulfonylamino, arylsulfonylamino, aminosulfonyl, alkylaminosulfonyl, dialkylaminosulfonyl, arylaminosulfonyl, aralkylaminosulfonyl, aminocarbonyl, arylaminocarbonyl, aralkylaminocarbonyl, amino, monosubstituted or disubstituted amino, and further wherein the aromatic or alicyclic ring in R^{g} is optionally substituted with one, two, or three R^{h} wherein R^{h} is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, amino, monosubstituted amino, dialkylamino, aryl, heteroaryl, cycloalkyl, carboxy, carboxamido, or alkoxycarbonyl; and

R⁴ is haloalkyl;

R^{4′} is hydrogen, alkyl, alkoxyalkyl, or haloalkyl; or

R³ and R^{4′} together with the carbon atom to which they are attached form cycloalkylene or heterocyclylalkylene wherein said cycloalkylene is optionally substituted with one or two substituents independently selected from alkyl, haloalkyl, hydroxy, or alkoxy and heterocyclylalkylene is optionally substituted with one to three substituents independently selected from alkyl, haloalkyl, hydroxy, alkoxy, carboxy, alkoxycarbonyl, alkylsulfonyl, aryl, heteroaryl, or hydroxyalkyl; or

a pharmaceutically acceptable salts thereof;

provided that when E is a group of formula (a), then: (i) R^{1a} is not hydrogen, alkyl, haloalkyl, cycloalkyl, or cycloalkylalkyl and (ii) R¹ and R^{1a} together with the carbon atoms to which they are attached do not form cycloalkylene or heterocyclylalkylene ring wherein said cycloalkylene or heterocyclylalkylene is optionally substituted with one or two R^{f} independently selected from alkyl, halo, hydroxyalkyl, keto, or —SO₂R where R is alkyl, cycloalkyl, cycloalkylalkyl, aryl, aralkyl, heteroaryl or heteroaralkyl and further where the aromatic or alicylic ring in R^{f} is optionally substituted with one, two, or three substitutents independently selected from alkyl, alkoxy, haloalkyl, haloalkoxy, hydroxy, halo, carboxy, or alkoxycarbonyl.

In a second aspect, this invention is directed to a pharmaceutical composition comprising a compound of Formula (I), individual stereoisomers or mixture of thereof, or a pharmaceutically acceptable salt thereof, in admixture with one or more suitable excipients.

In a third aspect, this invention is directed to a method for treating a disease in an animal mediated by cysteine proteases, in particular cathepsin S which method comprises administering to the animal a pharmaceutical composition comprising a therapeutically effective amount of a compound of Formula (I), individual isomer or mixture of isomers thereof; or a pharmaceutically acceptable salt thereof, in admixture with one or more suitable excipients.

In a fourth aspect, this invention is directed to intermediates of the formula (II): 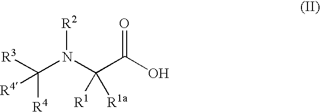
wherein:

R¹, R^{1a}, R², R³, R⁴ and R^{4′} are as defined in the Summary of the Invention and in the preferred embodiments below except that R^{1a} is not hydrogen, alkyl, haloalkyl, or cycloalkylalkyl or R¹ and R^{1a} together with the carbon atoms to which they are attached do not form cycloalkylene or heterocyclylalkylene ring.

Additional preferred group of compounds within intermediate (II) are those wherein:

R¹ is hydrogen;

R^{1a} is:

(a) aralkyl or heteroaralkyl, (preferably aralkyl) wherein the aromatic ring in R^{1a} is optionally substituted with one or two R^{e} independently selected from alkyl, haloalkyl, alkoxy, hydroxy, haloalkoxy, halo, nitro, cyano, carboxy, alkoxycarbonyl, amino, monosubstituted amino, disubstituted amino, or acyl, and an additional R^{e} selected from aryl, heteroaryl, heterocyclyl, or -(alkylene)ₘ—X⁷—R³⁵ [wherein X⁷ is —NR³⁶CO— (where R³⁶ is hydrogen, alkyl, or acyl and m is 0 or 1) and R³⁵ is aryl or heteroaryl] wherein the aromatic or alicyclic ring in R^{e} is optionally substituted with one, two, or three substituents independently selected from alkyl, alkoxy, alkoxyalkyl, alkylsulfonyl, alkylsulfonylalkyl, alkylaminosulfonyl, acyl, halo, haloalkyl, haloalkoxy, cyano, nitro, hydroxy, hydroxyalkyl, carboxy, alkoxycarbonyl, aryl optionally substituted with alkoxy or halo, aralkyl optionally substituted with alkoxy or halo, heteroaryl optionally substituted with alkoxy or halo, or heteroaralkyl optionally substituted with alkoxy or halo, amino, aminosulfonyl, alkylamino, dialkylamino, or alkynyl optionally substituted with hydroxy, aryl, or heteroaryl; and

R³ is aryl, heteroaryl, or heterocyclyl wherein the aromatic or alicyclic rings in R³ are optionally substituted by one, two, or three R^{g} independently selected from alkyl, halo, hydroxy, alkoxy, haloalkyl, haloalkoxy, oxo, cyano, nitro, acyl, acyloxy, aryl, aralkyl, heteroaryl, heteroaralkyl, cycloalkyl, cycloalkylalkyl, heterocyclyl, heterocyclylalkyl, aryloxy, benzyloxy, carboxy, alkoxycarbonyl, aryloxycarbonyl, carbamoyl, alkylthio, alkylsulfinyl, alkylsulfonyl, arylthio, arylsulfonyl, arylsulfinyl, alkoxycarbonylamino, aryloxycarbonylamino, alkylcarbamoyloxy, arylcarbamoyloxy, alkylsulfonylamino, arylsulfonylamino, aminosulfonyl, alkylaminosulfonyl, dialkylaminosulfonyl, arylaminosulfonyl, aralkylaminosulfonyl, aminocarbonyl, arylaminocarbonyl, aralkylaminocarbonyl, amino, monosubstituted or disubstituted amino, and further wherein the aromatic and alicyclic rings in R^{g} are optionally substituted with one, two, or three R^{h} wherein R^{h} is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, amino, monosubstituted amino, dialkylamino, aryl, heteroaryl, cycloalkyl, carboxy, carboxamido, or alkoxycarbonyl;

R⁴ is haloalkyl, preferably trifluoromethyl; and

R² and R^{4′} are hydrogen.

In a fifth aspect, this invention is directed to a method of treating a patient undergoing a therapy wherein the therapy causes an immune response, preferably a deleterious immune response, in the patient comprising administering to the patient a compound of Formula (I) or a pharmaceutically acceptable salt thereof. Preferably, the immune response is mediated by MHC class II molecules. The compound of this invention can be administered prior to, simultaneously, or after the therapy. Preferably, the therapy involves treatment with a biologic. Preferably, the therapy involves treatment with a small molecule.

Preferably, the biologic is a protein or an antibody, preferably a monoclonal antibody. More preferrably, the biologic is Remicade®, Refacto®, Referon-A®, Factor VIII, Factor VII, Betaseron®, Epogen®, Enbrel®, Interferon beta, Botox®, Fabrazyme®, Elspar®, Cerezyme®, Myobloc®, Aldurazyme®, Verluma®, Interferon alpha, Humira®, Aranesp®, Zevalin® or OKT3.

Preferably, the treatment involves use of heparin, low molecular weight heparin, procainamide or hydralazine.

In a sixth aspect, this invention is directed to a method of treating immune response in an animal that is caused by administration of a biologic to the animal which method comprises administering to the animal in need of such treatment a therapeutically effective amount of a compound of Formula (I) or a pharmaceutically acceptable salt thereof.

In a seventh aspect, this invention is directed to a method of conducting a clinical trial for a biologic comprising administering to an individual participating in the clinical trial a compound of Formula (I) or a pharmaceutically acceptable salt thereof with the biologic.

In an eighth aspect, this invention is directed to a method of prophylactically treating a person undergoing treatment with a biologic with a compound of Formula (I) or a pharmaceutically acceptable salt thereof to treat the immune response caused by the biologic in the person.

In a ninth aspect, this invention is directed to a method of determining the loss in the efficacy of a biologic in an animal due to the immune response caused by the biologic comprising administering the biologic to the animal in the presence and absence of a compound of Formula (I) or a pharmaceutically acceptable salt thereof.

In a tenth aspect, this invention is directed to a method of improving efficacy of a biologic in an animal comprising administering the biologic to the animal with a compound of Formula (I) or a pharmaceutically acceptable salt thereof.

In an eleventh aspect, this invention is directed to the use of a compound of Formula (I) or a pharmaceutically acceptable salt thereof for the manufacture of a medicament. Preferably, the medicament is for use in the treatment of a disease mediated by Cathepsin S.

In a twelfth aspect, this invention is directed to the use of a compound of Formula (I) or a pharmaceutically acceptable salt thereof for the manufacture of a medicament for combination therapy with a biologic, wherein the compound of this invention treats the immune response caused by the biologic. Preferably, the Cathepsin S inhibitor is administered prior to the administration of the biological agent. Preferably, the Cathepsin S inhibitor is administered concomitantly with the biological agent. Preferably, the Cathepsin S inhibitor is administered after the administration of the biological agent.

### DETAILED DESCRIPTION OF THE INVENTION

Definitions:

Unless otherwise stated, the following terms used in the specification and claims are defined for the purposes of this Application and have the following meanings.

“Alicyclic” means a moiety characterized by arrangement of the carbon atoms in closed non-aromatic ring structures e.g., cycloalkyl and heterocyclyl rings as defined herein.

“Alkyl” represented by itself means a straight or branched, saturated aliphatic radical containing one to six carbon atoms, unless otherwise indicated e.g., alkyl includes methyl, ethyl, propyl, isopropyl, butyl, sec-butyl, isobutyl, tert-butyl, and the like.

“Alkylene”, unless indicated otherwise, means a straight or branched, saturated aliphatic, divalent radical having the number of one to six carbon atoms, e.g., methylene (—CH₂—), ethylene (—CH₂CH₂—), trimethylene (—CH₂CH₂CH₂—), tetramethylene (—CH₂CH₂CH₂CH₂—) 2-methyltetramethylene (—CH₂CH(CH₃)CH₂CH₂—), pentamethylene (—CH₂CH₂CH₂CH₂CH₂—), and the like.

“Alkynyl” represented by itself means a straight or branched, aliphatic radical containing two to six carbon atoms, and one or two triple bonds unless otherwise indicated e.g., alkynyl includes ethynyl, propynyl, butynyl, and the like.

“Alkylcarbamoyloxy” refers to a radical —OCONHR where R is an alkyl group as defined herein e.g., methylcarbamoyloxy, ethylcarbamoyloxy, and the like.

“Alkylsulfonylamino” refers to a radical —NHSO₂R where R is an alkyl group as defined herein e.g., methylsulfonylamino, ethylsulfonylamino, and the like.

“Amino” means the radical —NH₂. Unless indicated otherwise, the compounds of the invention containing amino moieties include protected derivatives thereof. Suitable protecting groups for amino moieties include acetyl, tert-butoxycarbonyl, benzyloxycarbonyl, and the like.

“Aminosulfonyl” refers to a radical —SO₂NRR′ where R is hydrogen or alkyl and R′ is hydrogen, alkyl, aryl, aralkyl, alkoxyalkyl, or aminoalkyl as defined herein.

“Alkylaminosulfonyl” or “dialkylaminosulfonyl” refers to a radical —SO₂NHR and —SO₂NRR′ respectively, where R and R′ are independently alkyl group as defined herein e.g., methylaminosulfonyl, and the like.

“Alkylamino” or “dialkylamino” refers to a radical —NHR and —NRR′ respectively, where R and R′ are independently alkyl group as defined herein e.g., methylamino, dimethylamino, and the like.

“Alkoxy” refers to a radical —OR where R is an alkyl group as defined herein e.g., methoxy, ethoxy, and the like.

“Alkoxycarbonyl” refers to a radical —C(O)OR where R is an alkyl group as defined herein e.g., methoxycarbonyl, ethoxycarbonyl, and the like.

“Alkoxycarbonylalkyl” means a radical -(alkylene)-C(O)OR where R is alkyl as defined above e.g., methoxycarbonylmethyl, 2-, or 3-ethoxycarbonylpropyl, and the like.

“Alkoxycarbonylamino” refers to a radical —NHC(O)OR where R is an alkyl group as defined herein e.g., methoxycarbonyl, ethoxycarbonyl, and the like.

“Aminocarbonyl” refers to a radical —CONRR′ where R is hydrogen or alkyl and R′ is hydrogen, alkyl, aryl, aralkyl, alkoxyalkyl, or aminoalkyl as defined herein.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Alkoxyalkyloxyalkyl” refers to a radical -(alkylene)-O-(alkylene)-OR where R is an alkyl group as defined above, e.g., 2-methoxyethyloxymethyl, 3-methoxypropyloxyethyl, and the like.

“Aminoalkyl” means a linear monovalent hydrocarbon radical of one to six carbon atoms or a branched monovalent hydrocarbon radical of three to six carbons substituted with at least one, preferably one or two, —NRR′ where R is hydrogen, alkyl, or —COR^{a} where R^{e} is alkyl, and R′ is hydrogen or alkyl as defined herein e.g., aminomethyl, methylaminoethyl, dimethylaminoethyl, 1,3-diaminopropyl, acetylaminopropyl, and the like.

“Alkylthio” refers to a radical —SR where R is an alkyl group as defined herein e.g., methylthio, ethylthio, and the like.

“Alkylsulfinyl” refers to a radical —S(O)R where R is an alkyl group as defined herein e.g., methylsylfinyl, ethylsulfinyl, and the like.

“Alkylsulfonyl” refers to a radical —SO₂R where R is an alkyl group as defined herein e.g., methylsulfonyl, ethylsulfonyl, and the like.

“Alkylsulfonylalkyl” refers to a radical -(alkylene)-SO₂R where R is an alkyl group as defined herein e.g., methylsulfonylmethyl, ethylsulfonylmethyl, and the like.

“Alkylaminosulfonyl” refers to a radical —SO₂NHR where R is an alkyl group as defined herein e.g., methylaminisulfonyl, ethylaminosulfonyl, and the like.

“Acyl” means a radical —COR where R is hydrogen, alkyl, haloalkyl, aryl, aralkyl, heteroaryl, heteroaralkyl, or heterocyclyl as defined herein, e.g., formyl, acetyl, trifluoroacetyl, benzoyl, piperazin-1-ylcarbonyl, and the like.

“Acyloxy” means a radical —OCOR where R is alkyl, haloalkyl, aryl, aralkyl, heteroaryl, heteroaralkyl, or heterocyclyl as defined herein, e.g., acetyloxy, trifluoroacetyloxy, benzoyloxy, piperazin-1-ylcarbonyloxy, and the like.

“Animal” includes humans, non-human mammals (e.g., dogs, cats, rabbits, cattle, horses, sheep, goats, swine, deer, and the like) and non-mammals (e.g., birds, and the like).

“Aromatic” means a moiety wherein the constituent atoms make up an unsaturated ring system, all atoms in the ring system are sp² hybridized and the total number of pi electrons is equal to 4n+2.

“Aryl” means a monocyclic or fused bicyclic ring assembly containing 6 to 10 ring carbon atoms unless otherwise indicated, wherein each ring is aromatic e.g., phenyl or anthryl.

“Aralkyl” means a radical -(alkylene)-R where R is aryl as defined above e.g., benzyl, phenethyl, and the like.

“Aryloxy” means a radical —OR where R is aryl as defined above.

“Aryloxyalkyl” means the radical -(alkylene)-OR where R is aryl as defined above e.g., phenoxymethyl, 2-, or 3-phenoxymethyl, and the like

“Aryloxycarbonyl” means a radical C(O)OR where R is aryl as defined above e.g., phenyloxycarbonyl, and the like.

“Arylcarbamoyloxy” means a radical —OC(O)NHR where R is aryl as defined above e.g., phenylcarbamoyloxy, and the like.

“Arylthio” refers to a radical —SR where R is an aryl group as defined herein e.g., phenylthio, and the like.

“Arylsulfinyl” refers to a radical —SOR where R is an aryl group as defined herein e.g., phenylsulfinyl, and the like.

“Arylsulfonyl” refers to a radical —SO₂R where R is an aryl group as defined herein e.g., phenylsulfonyl, and the like.

“Aryloxycarbonylamino” refers to a radical —NHC(O)OR where R is an aryl group as defined herein e.g., phenoxycarbonylamino, and the like.

“Arylsulfonylamino” refers to a radical —NHSO₂R where R is an aryl group as defined above, unless otherwise stated e.g., phenylsulfonylamino, and the like.

“Arylaminosulfonyl” means the radical —SO₂NHR where R is aryl as defined above e.g., phenylaminosulfonyl, and the like.

“Aralkylaminosulfonyl” means the radical —SO₂NHR where R is aralkyl as defined above e.g., benzylaminosulfonyl, and the like.

“Arylaminocarbonyl” means a radical —CONHR where R is aryl as defined above e.g., phenylaminocarbonyl, and the like.

“Aralkylaminocarbonyl” means the radical —CONHR where R is aralkyl as defined above e.g., benzylaminocarbonyl, and the like.

“Biologic” means a therapeutic agent originally derived from living organisms for the treatment or management of a disease. Examples include, but are not limited to, proteins (recombinant and plasma derived), monoclonal or polyclonal, humanized or murine antibodies, toxins, hormones, vaccines, and the like. Several bBiologics are currently available for the treatment of a variousiety of diseases such as cancer, rheumatoid arthritis, and blood disorders such as and haemophilia.

“Carboxamide” or “carboxamido” means the radical —C(O)NH₂.

“Carbamoyl” means a radical —C(O)NRR′ where R and R′ are independently selected from hydrogen, alkyl, aryl, aralkyl, heteroaryl, heteroaralkyl, heterocyclyl or heterocyclylalkyl as defined herein provided one of R and R′ is not hydrogen.

“Carboxy” means the radical —C(O)OH.

“Carboxyalkyl” means a radical -(alkylene)-C(O)OH e.g., carboxymethyl, carboxyethyl, and the like.

“Cycloalkyl” means a monovalent saturated or partially unsaturated, monocyclic ring containing three to eight ring carbon atoms e.g., cyclopropyl, cyclobutyl, cyclopentyl, cyclohexyl, cyclohexenyl, 2,5-cyclohexadienyl, and the like.

“Cycloalkylalkyl” means a radical -(alkylene)-R where R is cycloalkyl as defined above e.g., cyclopropylmethyl, cyclobutylethyl, cyclobutylmethyl, and the like

“Cycloalkyloxy” means a radical —OR where R is cycloalkyl as defined above e.g., cyclobutyloxy, pentyloxy, hexyloxy, and the like.

“Cycloalkylene” means a divalent saturated or partially unsaturated monocyclic ring containing three to eight ring carbon atoms. For example, the instance wherein “R¹ and R^{1a} together with the carbon atom to which both R¹ and R^{1a} are attached form cycloalkylene” includes, but is not limited to, the following:

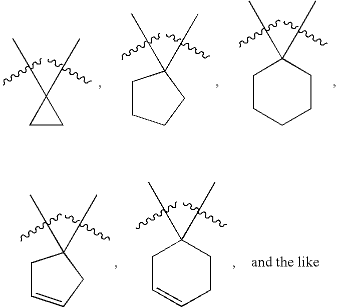

“Disubstituted amino” means a radical —NRR′ where R is alkyl, aryl, aralkyl, heteroaryl, heteroaralkyl, or heterocyclyl, and R′ is alkyl, aryl, aralkyl, heteroaryl, heteroaralkyl, cycloalkyl, heterocyclyl, cycloalkylalkyl, hydroxyalkyl, alkoxyalkyl, or acyl as defined herein. Representative examples include, but are not limited to, dimethylamino, methylphenylamino, benzylmethylamino, acetylmethylamino, and the like.

“Disease” specifically includes any unhealthy condition of an animal or part thereof and includes an unhealthy condition that may be caused by, or incident to, medical or veterinary therapy applied to that animal, i.e., the “side effects” of such therapy.

“Deleterious immune response” means an immune response that prevents effective treatment of a patient or causes disease in a patient. As an example, dosing a patient with a murine antibody either as a therapy or a diagnostic agent causes the production of human antimouse antibodies that prevent or interfere with subsequent treatments. The incidence of antibody formation versus pure murine monoclonals can exceed 70%. (see Khazaeli, M. B. et al. J. Immunother. 1994, 15, pp 42-52; Dillman R. O. et al. Cancer Biother. 1994, 9, pp 17-28; and Reinsberg, J. Hybridoma. 1995, 14, pp 205-208). Additional examples of known agents that suffer from deleterious immune responses are blood-clotting factors such as factor VIII. When administered to hemophilia A patients, factor VIII restores the ability of the blood to clot. Although factor VIII is a human protein, it still elicits an immune response in hemophiliacs as endogenous factor VIII is not present in their blood and thus it appears as a foreign antigen to the immune system. Approximately 29-33% of new patients will produce antibodies that bind and neutralize the therapeutically administered factor VIII (see Lusher J. M. Semin Thromb Hemost. 2002, 28(3), pp 273-276). These neutralizing antibodies require the administration of larger amounts of factor VIII in order to maintain normal blood clotting parameters; an expensive regimen of treatment in order to induce immune tolerance (see Briet E et al. Adv. Exp. Med. Bio. 2001, 489, pp 89-97). Another immunogenic example is adenoviral vectors. Retroviral therapy remains experimental and is of limited utility. One reason is that the application of a therapeutic virus generates an immune response capable of blocking any subsequent administration of the same or similar virus (see Yiping Yang et al. J. of Virology. 1995, 69, pp 2004-2015). This ensures that retroviral therapies must be based on the transient expression of a protein or the direct incorporation of viral sequence into the host genome. Directed research has identified multiple viral neutralizing epitopes recognized by host antibodies (see Hanne, Gahery-Segard et al. J. of Virology 1998. 72, pp 2388-2397) suggesting that viral modifications will not be sufficient to overcome this obstacle. This invention will enable a process whereby an adenoviral therapy will have utility for repeated application. Another example of an immunogenic agent that elicits neutralizing antibodies is the well-known cosmetic agent Botox. Botulin toxin protein, is purified from the fermentation of Clostridium botulinum. As a therapeutic agent, it is used for muscle disorders such as cervical dystonia in addition to cosmetic application. After repeated exposure patients generate neutralizing antibodies to the toxin that results in reduced efficacy (see Birklein F. et al. Ann Neurol. 2002, 52, pp 68-73 and Rollnik, J. D. et al. Neurol. Clin. Neurophysiol. 2001, 2001(3), pp 24). A “deleterious immune response” also encompasses diseases caused by therapeutic agents. A specific example of this is the immune response to therapy with recombinant human erythropoietin (EPO). Erythropoietin is used to stimulate the growth or red cells and restore red blood cell counts in patients who have undergone chemotherapy or dialysis. A small percentage of patients develop antibodies to EPO and subsequently are unresponsive to both therapeutically administered EPO and their own endogenous EPO (see Casadevall, N. et al., NEJM. 2002, 346, pp 469-475). They contract a disorder, pure red cell aplasia, in which red blood cell production is severely diminished (see Gershon S. K. et. al. NEJM. 2002, 346, pp 1584-1586). This complication of EPO therapy is lethal if untreated. Another specific example is the murine antibody, OKT3 (a.k.a., Orthoclone) a monoclonal antibody directed towards CD-3 domain of activated T-cells. In clinical trials 20-40% of patients administered OKT3 produce antibodies versus the therapy. These antibodies besides neutralizing the therapy also stimulate a strong host immune reaction. The immune reaction is severe enough that patients with high titers of human anti-mouse antibodies are specifically restricted from taking the drug (see Orthoclone package label). A final example is a human antibody therapeutic. Humira® is a monoclonal antibody directed against TNF and is used to treat rheumatoid arthritis patients. When taken alone ˜12% of patients develop neutralizing antibodies. In addition, a small percentage of patients given the drug also contract a systemic lupus erthematosus-like condition that is an IgG-mediated immune response induced by the therapeutic agent (see Humira package label).

Another example of “deleterious immune response” is a host reaction to small molecule drugs. It is known to those skilled in the art that certain chemical structures will conjugate with host proteins to stimulate immune recognition (see Ju. C. et al. 2002. Current Drug Metabolism 3, pp 367-377 and Kimber I. et al. 2002, Toxicologic Pathology 30, pp 54-58.) A substantial portion of this host reactions are IgG mediated. Specific “deleterious immune responses” that are IgG mediated and include: hemolytic anemia, Steven-Johnson syndrome and drug induced Lupus.

“Halo” means fluoro, chloro, bromo or iodo.

“Haloalkyl” means alkyl substituted by one or more, preferably one to five, “halo” atoms, as such terms are defined in this Application. Haloalkyl includes monohaloalkyl, dihaloalkyl, trihaloalkyl, perhaloalkyl and the like e.g. chloromethyl, dichloromethyl, difluoromethyl, trifluoromethyl, 2,2,2-trifluoroethyl, perfluoroethyl, 2,2,2-trifluoro-1,1-dichloroethyl, and the like).

“Haloalkoxy” refers to a radical —OR where R is haloalkyl group as defined above e.g., trifluoromethoxy, 2,2,2-trifluoroethoxy, difluoromethoxy, and the like.

“Heterocyclylalkylene” means a divalent heterocyclyl group, as defined in this Application, e.g., the instance wherein R¹ and R^{1a} together with the carbon atom to which both R¹ and R^{1a} are attached form heterocyclylalkylene” includes, but is not limited to, the following:

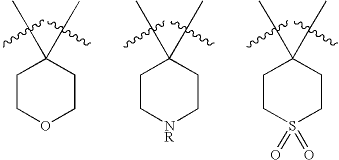

in which R is a substituent defined in the Summary of the Invention

“Heteroaryl” as a group or part of a group denotes an aromatic monocyclic or multicyclic moiety of 5 to about 10 ring atoms in which one or more, preferably one, two, or three, of the ring atom(s) is(are) selected from nitrogen, oxygen or sulfur, the remaining ring atoms being carbon. Representative heteroaryl rings include, but are not limited to, pyrrolyl, furanyl, thienyl, oxazolyl, isoxazolyl, thiazolyl, imidazolyl, triazolyl, tetrazolyl, pyridinyl, pyrimidinyl, pyrazinyl, pyridazinyl, indolyl, benzofuranyl, benzothienyl, benzimidazolyl, quinolinyl, isoquinolinyl, quinazolinyl, quinoxalinyl, pyrazolyl, and the like.

“Heteroaralkyl” means a radical -(alkylene)-R where R is heteroaryl as defined above e.g., pyridinylmethyl, 1- or 2-furanylethyl, imidazolylmethyl, and the like.

“Heteroaryloxyalkyl” means the radical -(alkylene)-OR where R is heteroaryl as defined above e.g., furanyloxymethyl, 2-, or 3-indolyloxyethyl, and the like.

“Heterocyclyl” means a saturated or partially unsaturated, mono or bicyclic radical of 5 or 6 ring atoms wherein one or more, preferably one, two, or three of the ring carbon atoms are replaced by a heteroatom selected from —N—, —N—, —O—, —S—, —SO—, or —S(O)₂— and further wherein one or two ring atoms are optionally replaced by a keto (—CO—) group. The heterocyclyl ring is optionally fused to cycloalkyl, aryl or heteroaryl ring as defined herein. Representative examples include, but are not limited to, imidazolidinyl, morpholinyl, thiomorpholinyl, thiomorpholino-1-oxide, thiomorpholino-1,1-dioxide, tetrahydropyranyl, tetrahydrothiopyranyl, 1-oxo-tetrahydrothiopyranyl, 1,1-dioxotetrathiopyranyl, indolinyl, piperazinyl, piperidyl, pyrrolidinyl, pyrrolinyl, quinuclidinyl, and the like.

“Heterocyclylalkyl” means a radical -(alkylene)-heterocyclyl as defined in this Application. Representative examples include, but are not limited to, imidazolidin-1-ylmethyl, morpholin-4-ylmethyl, thiomorpholin-4-ylmethyl, thiomorpholin-4-ylmethyl-1-oxide, indolinylethyl, piperazinylmethyl or ethyl, piperidylmethyl or ethyl, pyrrolidinylmethyl or ethyl, and the like.

“Hydroxy” means the radical —OH. Unless indicated otherwise, the compounds of the invention containing hydroxy radicals include protected derivatives thereof. Suitable protecting groups for hydroxy moieties include benzyl and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Isomers” mean compounds of Formula (I) having identical molecular formulae but differ in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center has two enantiomeric forms of opposite chirality is termed a “racemic mixture”. A compound that has more than one chiral center has 2¹⁻¹ enantiomeric pairs, where n is the number of chiral centers. Compounds with more than one chiral center may exist as ether an individual diastereomers or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). It is understood that the names and illustration used in this Application to describe compounds of Formula (I) are meant to be encompassed all possible stereoisomers.

“Keto or oxo” means the radical (═O).

“Monosubstituted amino” means a radical —NHR where R is alkyl, aryl, aralkyl, heteroaryl, heteroaralkyl, cycloalkyl, cycloalkylalkyl, hydroxyalkyl, alkoxyalkyl, or acyl as defined herein. Representative examples include, but are not limited to, methylamino, phenylamino, benzylamino, cycloalkylmethylamino, acetylamino, trifluoroacetyl, and the like.

“Nitro” means the radical —NO₂.

“Optional” or “optionally” or “may be” means that the subsequently described event or circumstance may or may not occur, and that the description includes instances where the event or circumstance occurs and instances in which it does not. For example, the phrase “wherein the aromatic ring R^{a} is optionally substituted with one or two substituents independently selected from alkyl.” means that the aromatic ring may or may not be substituted with alkyl in order to fall within the scope of the invention.

The present invention also includes N-oxide derivatives of a compound of Formula (I). N-oxide derivatives mean derivatives of compounds of Formula (I) in which nitrogens are in an oxidized state (i.e., N→O) e.g., pyridine N-oxide, and which possess the desired pharmacological activity.

The expression “ . . . wherein said alkylene chain in R⁴ or R⁶ is optionally substituted with one to six halo” in the Summary of the Invention refers to the alkylene chain in -alkylene-X¹—R²² and -alkylene-X²—R²⁵ respectively, being optionally substituted with halo.

The expression “ . . . wherein the aromatic or alicyclic ring in R⁶, R¹⁰, R^{1a}, or R³ is optionally substituted with one to three R^{a}, R^{d}, R^{e} or R^{g} respectively . . . ” refers to all the groups attached to R⁶, R¹⁰, R^{1a}, or R³ that contain an aromatic or alicyclic ring being optionally substituted with one to three R^{a}, R^{d}, R^{e} or R^{g} respectively, e.g., for R⁶ it includes the aromatic or alicyclic ring in alkyl, haloalkyl, alkoxy, hydroxy, haloalkoxy, halo, carboxy, alkoxycarbonyl, amino, monosubstituted amino, disubstituted amino, nitro, aryloxy, benzyloxy, acyl, or arylsulfonyl groups being optionally substituted with one to three R^{a}.

“Ring system” as used herein means a monocyclic, bridged, or fused bicyclic ring.

“Pathology” of a disease means the essential nature, causes and development of the disease as well as the structural and functional changes that result from the disease processes.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of Formula (I) which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methylsulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

The present invention also includes prodrugs of a compound of Formula (I). Prodrug means a compound that is convertible in vivo by metabolic means (e.g. by hydrolysis) to a compound of Formula (I). For example an ester of a compound of Formula (I) containing a hydroxy group may be convertible by hydrolysis in vivo to the parent molecule. Alternatively an ester of a compound of Formula (I) containing a carboxy group may be convertible by hydrolysis in vivo to the parent molecule. Suitable esters of compounds of Formula (I) containing a hydroxy group, are for example acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methylsulphonates, ethanesulphonates, benzenesulphonates, p-toluenesulphonates, cyclohexylsulphamates and quinates. Suitable esters of compounds of Formula (I) containing a carboxy group, are for example those described by F. J. Leinweber, Drug Metab. Res., 1987, 18, page 379. An especially useful class of esters of compounds of Formula (I) containing a hydroxy group, may be formed from acid moieties selected from those described by Bundgaard et al., J. Med. Chem., 1989, 32, 2503-2507, and include substituted (aminomethyl)-benzoates, for example, dialkylamino-methylbenzoates in which the two alkyl groups may be joined together and/or interrupted by an oxygen atom or by an optionally substituted nitrogen atom, e.g. an alkylated nitrogen atom, more especially (morpholino-methyl)benzoates, e.g. 3- or 4-(morpholinomethyl)-benzoates, and (4-alkylpiperazin-1-yl)benzoates, e.g. 3- or 4-(4-alkylpiperazin-1-yl)benzoates. “Protected derivatives” means derivatives of compounds of Formula (I) in which a reactive site or sites are blocked with protecting groups. Protected derivatives of compounds of Formula (I) are useful in the preparation of compounds of Formula (I) or in themselves may be active cathepsin S inhibitors. A comprehensive list of suitable protecting groups can be found in T. W. Greene, Protective Groups in Organic Synthesis, 3rd edition, John Wiley & Sons, Inc. 1999.

“Therapeutically effective amount” means that amount which, when administered to an animal for treating a disease, is sufficient to effect such treatment for the disease.

“Treatment” or “treating” means any administration of a compound of the present invention and includes:

(1) preventing the disease from occurring in an animal which may be predisposed to the disease but does not yet experience or display the pathology or symptomatology of the disease,

(2) inhibiting the disease in an animal that is experiencing or displaying the pathology or symptomatology of the diseased (i.e., arresting further development of the pathology and/or symptomatology), or

(3) ameliorating the disease in an animal that is experiencing or displaying the pathology or symptomatology of the diseased (i.e., reversing the pathology and/or symptomatology).

“Treatment” or “treating” with respect to combination therapy i.e., use with a biologic means any administration of a compound of the present invention and includes:

(1) preventing the immune response from occurring in an animal which may be predisposed to the immune response but does not yet experience or display the pathology or symptomatology of the immune response,

(2) inhibiting the immune response in an animal that is experiencing or displaying the pathology or symptomatology of the immune response (i.e., arresting further development of the pathology and/or symptomatology), or

(3) ameliorating the immune response in an animal that is experiencing or displaying the pathology or symptomatology of the immune response (i.e., reducing in degree or severity, or extent or duration, the overt manifestations of the immune response or reversing the pathology and/or symptomatology e.g., reduced binding and presentation of antigenic peptides by MHC class II molecules, reduced activation of T-cells and B-cells, reduced humoral and cell-mediated responses and, as appropriate to the particular immune response, reduced inflammation, congestion, pain, necrosis, reduced loss in the efficacy of a biologic agent, and the like).

### PREFERRED EMBODIMENTS

(I) While the broadest definition of this invention is set forth in the Summary of the Invention, certain compounds of Formula (I) are preferred. For example:

A. One preferred group of compounds is that wherein E is —C(R⁵)(R⁶)X¹ in which:

R⁵ is hydrogen or alkyl; and

R⁶ is hydrogen, alkyl, -(alkylene)-OR¹² (where R¹² is hydrogen, alkyl or haloalkyl), cycloalkyl, cycloalkylalkyl, aryl, aralkyl, heteroaryl, heteroaralkyl, heterocyclyl, heterocyclylalkyl wherein the aromatic or alicyclic ring in aryl, aralkyl, heteroaryl, heteroaralkyl, heterocyclyl or heterocyclylalkyl is optionally substituted with one, two, or three R^{a} independently selected from alkyl, haloalkyl, alkoxy, hydroxy, haloalkoxy, halo, carboxy, alkoxycarbonyl, amino, monosubstituted amino, disubstituted amino, or acyl.

Preferably, R⁵ is hydrogen;

R⁶ is alkyl, preferably ethyl; and

X¹ is —CHO, —C(O)R¹⁰, —C(O)CF₃, —C(O)CF₂CF₂R⁹—CH═CHS(O)₂R¹⁰, —C(O)CF₂C(O)NR¹⁰R¹¹, —C(O)C(O)NR¹⁰R¹¹, —C(O)CH₂OR¹⁰, —C(O)CH₂N(R¹¹)SO₂R¹⁰, —C(O)C(O)N(R¹¹)(CH₂)₂OR¹¹, —C(O)C(O)N(R¹¹)(CH₂)₂NHR¹¹ or —C(O)C(O)R¹⁰; wherein R¹⁰ is alkyl, aryl, aralkyl, heteroaryl, heteroaralkyl, cycloalkylalkyl or heterocyclylalkyl wherein the aromatic ring is optionally substituted with R^{d} selected from heteroaryl, aryl, or alkyl, R¹¹ is hydrogen or alkyl and R⁹ is halo.

Preferably, E is —CHR⁶C(O)R¹⁰ where R⁶ is alkyl, preferably ethyl, propyl, or butyl, more preferably ethyl, and R¹⁰ is heteroaryl optionally substituted with one or two R^{d} independently selected from alkyl, haloalkyl, alkoxy, cycloalkyl, hydroxy, haloalkoxy, halo, carboxy, alkoxycarbonyl, aryl, heteroaryl, amino, monosubstituted amino, disubstituted amino, or acyl wherein the aromatic or alicyclic ring in R^{d} is optionally substituted with one, two, or three substitutents independently selected from alkyl, haloalkyl, alkoxy, haloalkoxy, halo, hydroxy, carboxy, alkoxycarbonyl, amino, alkylamino, or dialkylamino, more preferably R¹⁰ is benzoxazol-2-yl, 4-azabenzoxazol-2-yl, 2-pyridin-3-yl-[1,3,4]-oxadiazol-5-yl, 2-pyridin-4-yl-[1,3,4]-oxadiazol-5-yl, 2-ethyl-[1,3,4]-oxadiazol-5-yl, 2-isopropyl-[1,3,4]-oxadiazol-5-yl, 2-tert-butyl-[1,3,4]-oxadiazol-5-yl, 2-phenyl-[1,3,4]-oxadiazol-5-yl, 2-methoxymethyl-[1,3,4]-oxadiazol-5-yl, 2-furan-2-yl-[1,3,4]-oxadiazol-5-yl, 2-thien-2-yl-[1,3,4]-oxadiazol-5-yl, 2-(4-methoxyphenyl)-[1,3,4]-oxadiazol-5-yl, 2-(2-methoxyphenyl)-[1,3,4]-oxadiazol-5-yl, 2-(3-methoxyphenyl)-[1,3,4]-oxadiazol-5-yl, 2-(2-trifluoromethoxyphenyl)-[1,3,4]-oxadiazol-5-yl, 2-(3-trifluoromethoxyphenyl)-[1,3,4]-oxadiazol-5-yl, 2-(4-trifluoromethoxyphenyl)-[1,3,4]oxadiazol-5-yl, 2-(4-dimethylaminophenyl)-[1,3,4]-oxadiazol-5-yl, pyradizin-3-yl, pyrimidin-2-yl, 3-phenyl-[1,2,4]-oxadiazol-5-yl, 3-ethyl-[1,2,4]-oxadiazol-5-yl, 3-cyclopropyl-[1,2,4]-oxadiazol-5-yl, 3-thien-3-yl-[1,2,4]-oxadiazol-5-yl, 3-pyridin-4-yl-[1,2,4]-oxadiazol-5-yl, 3-pyridin-2-yl-[1,2,4]-oxadiazol-5-yl, 5-ethyl-[1,2,4]-oxadiazol-3-yl, 5-phenyl-[1,2,4]-oxadiazol-3-yl, 5-thien-3-yl-[1,2,4]-oxadiazol-3-yl, 5-trifluoromethyl-[1,2,4]-oxadiazol-3-yl, 5-pyridin-4-yl-[1,2,4]-oxadiazol-3-yl, or 5-phenyloxazol-2-yl, most preferably benzoxazol-2-yl.

B. Another preferred group of compounds is that wherein E is —C(R⁵)(R⁶)X¹ in which R⁵ and R⁶ taken together with the carbon atom to which both R⁵ and R⁶ are attached form cycloalkylene or heterocyclylalkylene, preferably cyclopropylene, cyclopentylene, cyclohexylene, thiomorpholinyl-1-dioxide, tetrahydropyran-4-yl, tetrahydrothiopyran-4-yl, tetrahydropyran-4-yl-1-oxide, tetrahydropyran-4-yl,-1,1-dioxide, or piperidin-4-yl wherein the nitrogen atom is optionally substituted with alkyl or hydroxy, preferably tetrahydrothiopyran-4-yl-1,1-dioxide, and X¹ is —CHO, —C(O)R¹⁰, —C(O)CF₃, —C(O)CF₂CF₂R⁹—CH═CHS(O)₂R¹⁰, —C(O)CF₂C(O)NR¹⁰R¹¹, —C(O)C(O)NR¹⁰R¹¹, —C(O)CH₂OR¹⁰, —C(O)CH₂N(R¹¹)SO₂R¹⁰, —C(O)C(O)N(R¹¹)(CH₂)₂OR¹¹, —C(O)C(O)N(R¹¹)(CH₂)₂NR¹ or —C(O)C(O)R¹⁰. More preferably, —C(O)C(O)NR¹⁰R¹¹ where R¹¹ is hydrogen and R¹⁰ is benzyl.
C. Yet another preferred group of compounds is that wherein E is a group of formula (a): 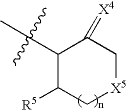
in which:

n is 0, 1, or 2, X⁴ is —NR²²—, —O— or —S— where R²² is hydrogen, alkyl, or alkoxy; X⁵ is —O—, —S(O)₂—, —S— or —NR²³ where R²³ is selected from hydrogen, alkyl, —S(O)R²⁴, —C(O)OR²⁶, or acyl where R²⁴ is alkyl, haloalkyl, cycloalkyl, cycloalkylalkyl, heterocyclyl, heterocyclylalkyl, aryl, aralkyl, heteroaryl, or heteroaralkyl and R²⁶ is hydrogen or alkyl. Preferably, X⁴ is —O—, n is 0 or 1, and X⁵ is —O—.

(a) Within the above preferred and more preferred groups (A-C), an even more preferred group of compounds is that wherein:

R^{1a} is alkyl, cycloalkyl, aralkyl, heteroaralkyl, cycloalkylalkyl, heterocyclylalkyl, or -alkylene-X—R³² (wherein X is —NR³³—, —O—, —S(O)ₙ₄—, —CO—, —COO—, —OCO—, —NR³³CO—, —CONR³³—, —NR³³SO₂—, —SO₂NR³³—, —NR³³COO—, —OCONR³³—, —NR³³CONR³⁴, or —NR³³SO₂NR³⁴— where R³³ and R³⁴ are independently hydrogen, alkyl, or acyl, and n4 is 0-2, and R³² is hydrogen, alkyl, haloalkyl, cycloalkyl, cycloalkylalkyl, heterocyclyl, aryl, aralkyl, heteroaryl, heteroaralkyl, heterocyclyl, or heterocyclylalkyl) wherein said alkylene chain is optionally substituted with one to six halo and wherein the aromatic or alicyclic ring in R^{1a} is optionally substituted with one, two, or three R^{e} independently selected from alkyl, haloalkyl, alkoxy, hydroxy, haloalkoxy, halo, nitro, cyano, carboxy, alkoxycarbonyl, aryl, heteroaryl, cycloalkyl, cycloalkylalkyl, aralkyl, heteroaralkyl, amino, monosubstituted amino, disubstituted amino, or acyl; and

R¹ and R² are hydrogen.

Preferably, R^{1a} is 2-methylpropyl, 2,2-dimethylpropyl, 3,3-dimethylbutyl, 3-methylbutyl, 2,2,3-trimethylbutyl, 3,3-dimethylpentyl, 3-ethyl-3-methylpentyl, n-butyl, 2-methylbutyl, or 1-methylpropyl.

Preferably, R^{1a} is 4,4-dimethylcyclohexylmethyl, 4-ethyl-4-methylcyclohexylmethyl, 4,4-diethylcyclohexylmethyl, 3,3-dimethylcyclohexylmethyl, 3,5-dimethylcyclohexylmethyl, cycloheptylmethyl, cyclooctylmethyl, 2-cyclohexylethyl, 2-cyclohexyl-2-methylpropyl, 2-(1-methylcyclohexyl)ethyl, 2-(1-methylcyclopropyl)ethyl, 2-(1-methylcyclopropyl)-2-methyl-propyl, 2-cyclopentylethyl, 2-cyclopentyl-2-methylpropyl, 4-isopropyl-4-methylcyclohexyl-methyl, 2-methylcyclohexylmethyl, 4-methoxycyclohexylmethyl, 1-methylcyclopentylmethyl, cyclohexyl, cyclohexylmethyl, 1,4-dimethylcyclopentylmethyl, cyclohexylethyl, cyclohexylmethyl, cyclopentylmethylmethylcyclohexylmethyl, 1-methylcyclopentylmethyl, or 1-benzylcyclopropylmethyl, preferably 1-methylcyclopentylmethyl.

Preferably, R^{1a} is 2-bicylo[2.2.1]hep-3-tylethyl, 8-methyl-8-aza-bicyclo[3.2.1]oct-3-ylmethyl, bicyclo[3.2.1]oct-3-ylmethyl, bicyclo[3.1.1]hept-3-ylmethyl, 6,6-dimethylbicyclo[3.1.1]hept-3-ylmethyl, 6,6-dimethylbicyclo[3.1.1]hept-4-ylmethyl, 2-bicyclo[2.2.1]hept-1-ylethyl, or bicyclo[2.2.1]hept-2-ylethyl.

Preferably, R^{1a} is tetrahydronaphthylmethyl, benzyl, 4-methoxybenzyl, 4-dimethylaminobutyl, 2-dimethylaminocarbonylethyl, dimethylaminocarbonylmethyl, methoxycarbonylmethyl, 3,4-dichlorobenzyl, 2-chlorobenzyl, 4-ethoxybenzyl, 4-nitrobenzyl, biphen-4-ylmethyl, naphth-1-ylmethyl, naphth-2-ylmethyl, 4-chlorobenzyl, 3-chlorobenzyl, 4-fluorobenzyl, 2-phenethyl, 4-hydroxybenzyl, 2-(4-hydroxyphenyl)ethyl, 2,6-difluorobenzyl, 2,2-difluoro-3-phenylpropyl, 2,2-dichloro-3-phenylpropyl, biphenyl-3-ylmethyl, naphth-2-yl, 3-phenylpropyl, 2,2-difluoro-3-phenylpropyl, or 2,2-dimethyl-3-phenylpropyl.

Preferably, R^{1a} is ethylthiomethyl, ethylsulfinylmethyl, ethylsulfonylmethyl, isopropylthiomethyl, 2-methylthioethyl, 2-methylsulfinylethyl, 2-methysulfonylethyl, 2-methylpropylsulfonylmethyl, isobutylsulfanylmethyl, tert-butylthiomethyl, benzenesulfonylmethyl, 2-phenylsulfanylethyl, 2-phenylsulfonylethyl, naphth-2-ylmethanesulfonylmethyl, biphenyl-2-ylmethanesulfonylmethyl, biphenyl-4-ylmethanesulfonylmethyl, phenylmethanesulfanylmethyl, phenylmethane-sulfinylmethyl, phenylmethanesulfonylmethyl, 2-phenylmethanesulfonylethyl, 4-tert-butylphenylmethanesulfonylmethyl, 2-fluorophenylmethanesulfanylmethyl, 2-fluoro-phenylmethanesulfonylmethyl, 3-fluorophenylmethanesulfonylmethyl, 4-fluorophenylmethanesulfonylmethyl, 2-chlorophenylmethanesulfanylmethyl, 2-chloro-phenylmethanesulfonylmethyl, 3-chlorophenylmethanesulfonylmethyl, 4-chlorophenyl-methanesulfonylmethyl, 2-methoxyphenylmethanesulfonylmethyl, 4-methoxyphenyl-methanesulfonylmethyl, 2-trifluoromethoxyphenylmethanesulfonylmethyl, 3-trifluoromethoxyphenylmethanesulfonylmethyl, 4-trifluoromethoxyphenylmethanesulfonyl-methyl, 2-trifluoromethylphenylmethanesulfanylmethyl, 2-trifluoromethylphenylmethane-sulfonylmethyl, 3-trifluoromethylphenylmethanesulfonylmethyl, 4-trifluoromethylphenyl-methanesulfonylmethyl, 2-cyanophenylmethanesulfanylmethyl, 2-cyanophenylmethane-sulfonylmethyl, 3-cyanophenylmethanesulfonylmethyl, 2-bromophenylmethanesulfonylmethyl, 2-nitrophenylmethanesulfanylmethyl, 2-nitro-phenylmethanesulfonylmethyl, 2-methylphenyl-methanesulfonylmethyl, 3-methylphenylmethanesulfonylmethyl, 4-methylphenylmethane-sulfonylmethyl, 2-(4-trifluoromethoxy-benzenesulfonyl)ethyl, 2-(3-trifluoromethoxybenzenesulfonyl)ethyl, 2-(2-trifluoromethoxybenzenesulfonyl)ethyl, 2-difluoromethoxyphenylmethane-sulfonylmethyl, 3-difluoromethoxyphenylmethane-sulfonylmethyl, 4-difluoromethoxy-phenylmethane-sulfonylmethyl, 2-(4-difluoromethoxybenzenesulfonyl)ethyl, 2-(2-difluoromethoxybenzene-sulfonyl)ethyl, 2-(3-difluoromethoxybenzenesulfonyl)ethyl, 3-chloro-2-fluorophenylmethane-sulfonylmethyl, 3,5-dimethylphenylmethanesulfonylmethyl, 3,5-bis-trifluoromethylphenylmethane-sulfonylmethyl, 2,5-difluorophenylmethanesulfonylmethyl, 2,6-difluorophenylmethanesulfonylmethyl, 2,3-difluorophenylmethane-sulfonylmethyl, 3,4-difluorophenylmethanesulfonylmethyl, 2,4-difluorophenyl-methanesulfonylmethyl, 2,5-dichlorophenylmethanesulfonylmethyl, 3,4-dichlorophenylmethanesulfonylmethyl, 2,6-dichlorophenylmethanesulfonylmethyl, 2-fluoro-3-methylphenylmethanesulfonyl-methyl, 4-fluoro-2-trifluoromethoxyphenyl-methanesulfonylmethyl, 2-fluoro-6-trifluoromethylphenylmethanesulfonylmethyl, 2-fluoro-3-trifluoromethylphenyl-methanesulfonylmethyl, 2-fluoro-4-trifluoromethyl-phenylmethanesulfonylmethyl, 2-fluoro-5-trifluoromethyl-phenylmethanesulfonylmethyl, 4-fluoro-3-trifluoromethyl-phenylmethanesulfonylmethyl, 2-chloro-5-trifluoromethyl-phenylmethane-sulfonylmethyl, 2,4,6-trifluorophenylmethanesulfonylmethyl, 2,4,5-trifluorophenylmethanesulfonylmethyl, 2,3,4-trifluorophenylmethanesulfonylmethyl, 2,3,5-trifluorophenylmethanesulfonylmethyl, 2,5,6-trifluorophenylmethanesulfonyl-methyl, 3,4,5-trimethoxyphenylmethanesulfonylmethyl, pyridin-2-ylmethanesulfonylmethyl, pyridin-3-ylmethanesulfonylmethyl, pyridin-4-ylmethanesulfonylmethyl, 2-(pyridin-2-ylsulfonyl)ethyl, 2-(pyridin-4-ylsulfonyl)ethyl, oxypyridin-2-ylmethanesulfonylmethyl, cyclohexylmethyl, cyclohexylmethanesulfanylmethyl, cyclohexylsulfinylthiomethyl, cyclohexylmethane-sulfonylmethyl, 2-cyclohexylethanesulfonyl, cyclohexylmethanesulfonylmethyl, cyclopropylmethanesulfonylmethyl, thiophene-2-sulfonylmethyl, 5-chlorothien-2-ylmethane-sulfonylmethyl, or 3,5-dimethyl-isoxazol-4-ylmethanesulfonylmethyl, preferably 2-(difluoromethoxy)phenylmethane-sulfonylmethyl.

Preferably, R^{1a} is 1-ethoxycarbonylpiperidin-4-ylmethyl, 1-methylpiperidin-4-ylmethyl, 2-tetrahydropyran-4-ylethyl, pyrrolidin-1-ylmethyl, piperidin-1-ylmethyl, morpholin-4-ylmethyl, 1-morpholin-4-ylethyl, thiomorpholin-4-ylmethyl, 1-oxo-thiomorpholin-4-ylmethyl, 1,1-dioxothiomorpholin-4-ylmethyl, tetrahydrothiopyran-4-ylmethyl, 1-oxotetrahydrothiopyran-4-ylmethyl, 1,1-dioxotetrahydrothiopyran-4-ylmethyl, 1-methylpiperazin-4-ylmethyl, benzyloxymethyl, ethoxymethyl, isopropyloxymethyl, 2-dimethylaminoethyl, 2-piperidin-1-ylethyl, 2-pyrrolidin-1-ylethyl, tert-butyloxymethyl, imidazol-4-ylmethyl, indol-3-ylmethyl, 2-pyrrolidin-1-ylcarbonylethyl, pyrrolidin-1-ylcarbonylmethyl, indol-2-ylmethyl, 1-benzylimidazol-4-ylmethyl, 4-ethyl-4-methylpiperidin-1-ylmethyl, indol-1-ylmethyl, 1-methylpiperidin-2-ylmethyl, 2,2,-difluoro-3-thien-2-ylmethyl, or pyridin-4-ylmethyl.

More preferably, R^{1a} is cyclohexyl, 2-cyclohexylethyl, cyclohexylmethyl, tert-butylmethyl, 1-methylcyclohexylmethyl, 1-methylcyclopentylmethyl, 2,2-difluoro-3-phenylpropyl, 2,2-dichloro-3-phenylpropyl, 2,2,2-trichloroethyl, 2,2-dichloroethyl, 1,4-dimethylcyclopentylmethyl, 2,2-dimethyl-3-phenylpropyl, 1-benzylcyclopropylmethyl, 2-(1,1-difluoromethoxy)phenylmethane-sulfonylmethyl, 2-(1,1-difluoromethoxy)phenylmethaneoxy-methyl, pyridin-4-ylmethyl, phenylmethanesulfonylmethyl, pyridin-2-ylmethanesulfonylmethyl, pyridin-4-ylmethanesulfonyl-methyl, 2-methylpropylsulfonylmethyl, cyclopropylmethanesulfonylmethyl, pyridin-3-ylmethane-sulfonylmethyl, 2,6-difluorophenylmethanesulfonylmethyl, 2-pyridin-2-ylsulfonylethyl, 2-phenylsulfonylethyl, benzyloxymethyl, 2,2-dimethylpropyl, cyclopentylmethyl, morpholin-4-ylmethyl, 5-bromothien-2-ylmethyl, pyridin-4-ylmethyl, 2-chlorobenzyl, or 4-fluorobenzyl; most preferably 1-methylcyclopentylmethyl; and

R¹ and R² are hydrogen.

(b) Yet another more preferred group of compounds within groups (A-C) is that wherein R¹ and R^{1a} together with the carbon atoms to which they are attached form cycloalkylene or heterocyclylalkylene, preferably 3,3-dimethylcyclobutyl, cyclohexyl, cyclopentyl, cyclooctyl, tetrahydrothiopyran-1,1-dioxide, or piperidin-4-yl wherein the nitrogen atom at the 1-position of the piperidinyl ring is optionally substituted with R^{f} where R^{f} is alkyl or —SO₂R where is alkyl, cycloalkyl, cycloalkylalkyl, aryl, aralkyl, heteroaryl or heteroaralkyl where the rings in R^{f} are optionally substituted with one, two, or three substitutents independently selected from alkyl, alkoxy, haloalkyl, haloalkoxy, hydroxy, halo, or carboxy.

(1) Within the above preferred, more preferred, and even more preferred groups above, a particularly preferred group of compounds is that wherein:

R³ is alkyl, cycloalkyl, phenyl, benzyl, naphthyl, alkylSO₂alkyl, cycloalkylSO₂alkyl, arylSO₂alkyl, pyrrolidinyl, piperidinyl, morpholinyl, thiomorpholinyl, piperazinyl, indolinyl, pyranyl, thiopyranyl, furanyl, thienyl, pyrrolyl, oxazolyl, thiazolyl, imidazolyl, pyridinyl, isoxazolyl, pyrimidinyl, pyrazinyl, pyridazinyl, indolyl, quinolinyl, benzofuranyl, benzthienyl, benzimidazolyl, benzthiazolyl, benzoisoxazolyl, or benzoxazolyl; wherein the aromatic or alicyclic ring in R³ is optionally substituted by one, two, or three R^{g};

each R^{g} is independently alkyl, halo, hydroxy, oxo, carboxy, cyano, nitro, cycloalkyl, phenyl, naphthyl, pyrrolidinyl, piperidinyl, morpholinyl, thiomorpholinyl, piperazinyl, furanyl, thienyl, oxazolyl, thiazolyl, imidazolyl, triazolyl, tetrazolyl, pyridinyl, pyrimidinyl, pyrazinyl, indolyl, benzofuranyl, benzothienyl, benzimidazolyl, benzthiazolyl, benzoxazolyl, quinolinyl, isoquinolinyl, quinazolinyl, quinoxalinyl, alkoxy, —COR (where R is alkyl), —OC(O)R (where R is alkyl or aryl), aryloxy, benzyloxy, alkoxycarbonyl, aryloxycarbonyl, carbamoyl wherein the nitrogen atom may be independently mono or di-substituted by alkyl, aryl, pyrrolidinyl, piperidinyl, morpholinyl, thiomorpholinyl, piperazinyl, furanyl, thienyl, oxazolyl, thiazolyl, imidazolyl, triazolyl, tetrazolyl, pyridinyl, pyrimidinyl, pyrazinyl, indolyl, benzofuranyl, benzothienyl, benzimidazolyl, benzthiazolyl, quinolinyl, isoquinolinyl, quinazolinyl or quinoxalinyl, —NHCOR (where R is alkyl or aryl), alkylthio, arylthio, alkylsulfinyl, alkylsulfonyl, arylsulfinyl, arylsulfonyl, alkoxycarbonylamino, aryloxycarbonylamino, alkylcarbamoyloxy, arylcarbanoyloxy, alkylsulfonylamino, arylsulfonylamino, alkylaminosulfonyl, arylaminosulfonyl, amino wherein the nitrogen atom may be independently mono or di-substituted by alkyl, aryl, pyrrolidinyl, piperidinyl, morpholinyl, thiomorpholinyl, piperazinyl, furanyl, thienyl, oxazolyl, thiazolyl, imidazolyl, triazolyl, tetrazolyl, pyridinyl, pyrimidinyl, pyrazinyl, indolyl, benzofuranyl, benzothienyl, benzimidazolyl, benzthiazolyl, quinolinyl, isoquinolinyl, quinazolinyl or quinoxalinyl where the aromatic or alicyclic rings in R^{g} may be further optionally substituted by one, two or three R^{h} independently selected from alkyl, alkoxy, haloalkyl, haloalkoxy, halo, hydroxy, carboxy, carboxamido, cyano, nitro, aryl or cycloalkyl.

Preferably, R³ is methyl, ethyl, isopropyl, cyclopropyl, cyclopentyl, cyclohexyl, phenyl, benzyl, naphthyl, pyrrolidinyl, piperidinyl, morpholinyl, thiomorpholinyl, piperazinyl, furanyl, thienyl, thiazolyl, imidazolyl, pyridinyl, or pyrazinyl wherein the aromatic or alicylic rings in R³ are optionally substituted with one, two, or three R^{g} independently selected from methyl, ethyl, fluoro, chloro, bromo, iodo, hydroxy, oxo, carboxy, cyano, nitro, cyclopropyl, phenyl, pyrrolidinyl, piperidinyl, morpholinyl, thiomorpholinyl, piperazinyl, thienyl, imidazolyl, methoxy, acetyl, acetoxy, phenoxy, benzyloxy, methoxycarbonyl, phenoxycarbonyl, carbamoyl wherein the nitrogen atom is mono or disubstituted independently with methyl, ethyl or phenyl, acetylamino, benzoylamino, methylthio, phenylthio, phenylsulfonyl, methylsulfonyl, methoxycarbonylamino, phenoxycarbonylamino, methylcarbamoyloxy, phenylcarbamoyloxy, methylsulfonylamino, phenylsulfonylamino, methylaminosulfonyl, phenylaminosulfonyl, amino wherein the nitrogen atom is mono or disubstituted independently with methyl or phenyl wherein the aromatic or alicyclic rings in R^{g} are further optionally substituted with one, two, or three R^{h} independently selected from methyl, cyclopropyl, phenyl, methoxy, fluoro, chloro, hydroxy, or carboxy.

Even more preferably, R³ is phenyl, naphthyl, pyrrolidinyl, piperidinyl, morpholinyl, thiomorpholinyl, furanyl, thienyl, thiazolyl, imidazolyl, pyridinyl, or pyrazinyl wherein the aromatic or alicyclic rings in R³ are optionally substituted with one, two, or three R^{g} independently selected from methyl, fluoro, chloro, phenyl, thienyl, methoxy, acetyl, acetoxy, phenoxy, benzyloxy, methoxycarbonyl, carbamoyl wherein the nitrogen atom is mono or disubstituted independently with methyl or phenyl, acetylamino, methylthio, phenylthio, phenylsulfonyl, methylsulfonyl, methoxycarbonylamino, methylcarbamoyloxy, phenylcarbamoyloxy, methylsulfonylamino, phenylsulfonylamino, or amino wherein the nitrogen atom is mono or disubstituted independently with methyl or phenyl. Most preferably, R³ is phenyl, 4-methoxyphenyl, 3-phenoxyphenyl, 4-chlorophenyl, 4-fluorophenyl, 2-fluorophenyl, 2-fluoro-4-chlorophenyl, naphthyl, piperidin-4-yl, morpholin-4-yl, furanyl, thienyl, pyridin-4-yl, or pyrazinyl.

(2) Within the above preferred, more preferred, and even more preferred groups above, another particularly preferred group of compounds is that wherein:

R³ is hydrogen or haloalkyl, preferably hydrogen or trifluoromethyl.

Within the above preferred, more preferred, even more preferred groups above, and particularly preferred groups 1 and 2, most preferred group of compounds is that wherein R⁴ is trifluoromethyl or 2,2,2-trifluoroethyl, more preferably trifluoromethyl; and

R^{4′} is hydrogen.

(3) Within the above preferred, more preferred, and even more preferred groups above, a particularly preferred group of compounds is that wherein:

R³ and R^{4′} together with the carbon to which they are attached from cycloalkylene, preferably cyclopentylene, cyclopent-1-enylene, cyclohexylene, cyclohex-1-enylene.

(4) Within the above preferred, more preferred, and even more preferred groups above, a particularly preferred group of compounds is that wherein:

R³ and R^{4′} together with the carbon to which they are attached from heterocyclylalkylene, preferably tetrahydropyran-4-yl or 3,6-dihydro-2H-pyran-4-yl.

Within the above preferred, more preferred, even more preferred groups above, and particularly preferred groups 3 and 4, most preferred group of compounds is that wherein R⁴ is trifluoromethyl or 2,2,2-trifluoroethyl, more preferably trifluoromethyl.

(c) Within the above preferred and more preferred groups (AC), another even more preferred group of compounds is that wherein:

R³ is phenyl, naphthyl, pyrrolidinyl, piperidinyl, morpholinyl, thiomorpholinyl, furanyl, thienyl, thiazolyl, imidazolyl, pyridinyl, or pyrazinyl wherein the aromatic or alicyclic rings in R³ are optionally substituted with one, two, or three R^{g} independently selected from methyl, fluoro, chloro, phenyl, thienyl, methoxy, acetyl, acetoxy, phenoxy, benzyloxy, methoxycarbonyl, carbamoyl wherein the nitrogen atom is mono or disubstituted independently with methyl or phenyl, acetylamino, methylthio, phenylthio, phenylsulfonyl, methylsulfonyl, methoxycarbonylamino, methylcarbamoyloxy, phenylcarbamoyloxy, methylsulfonylamino, phenylsulfonylamino, amino wherein the nitrogen atom is mono or disubstituted independently with methyl or phenyl. Most preferably, R³ is phenyl, 4-methoxyphenyl, 3-phenoxyphenyl, 4-chlorophenyl, 4-fluorophenyl, 2-fluorophenyl, 2-fluoro-4-chlorophenyl, naphthyl, piperidin-4-yl, morpholin-4-yl, furanyl, thienyl, pyridin-4-yl, or pyrazinyl. Particularly preferably R³ is morpholin-4-yl.

(d) Within the above preferred and more preferred groups (A-C), another even more preferred group of compounds is that wherein:

R³ is hydrogen or haloalkyl, preferably hydrogen or trifluoromethyl.

Within the above preferred groups (c) and (d), and a more preferred group of compounds is that wherein R⁴ is trifluoromethyl or 2,2,2-trifluoroethyl, more preferably trifluoromethyl; and

R⁴ is hydrogen.

(e) Within the above preferred and more preferred groups (A-C), an even more preferred group of compounds is that wherein R³ and R^{4′} together with the carbon to which they are attached from cycloalkylene, preferably cyclopentylene, cyclopent-1-enylene, cyclohexylene, cyclohex-1-enylene.

(f) Within the above preferred and more preferred groups (A-C), an even more preferred group of compounds is that wherein:

R³ and R^{4′} together with the carbon to which they are attached from heterocyclylalkylene, preferably tetrahydropyran-4-yl or 3,6-dihydro-2H-pyran-4-yl.

Within the above preferred, more preferred groups (e) and (f) above, and particularly preferred group of compounds is that wherein R⁴ is trifluoromethyl or 2,2,2-trifluoroethyl, more preferably trifluoromethyl.

Within these preferred, more preferred and particularly preferred groups (c)-(f), most preferred groups are those wherein R¹, R^{1a} and R² are as described in preferred embodiments (a) and (b) above.

A number of different preferences have been given above, and following any one of these preferences results in a compound of this invention that is more presently preferred than a compound in which that particular preference is not followed. However, these preferences are generally independent; and following more than one of these preferences may result in a more presently preferred compound than one in which fewer of the preferences are followed.

### General Synthetic Scheme

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 140 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1999.

Compounds of Formula (I) where E is —C(R⁵)(R⁶)C(R⁷)(R⁸)R¹⁰ and where R¹, R^{1a}, R², R³, R⁴, R⁵, R⁶, R⁷ and R⁸ are as defined in the Summary of the Invention and R^{4′} is hydrogen can be prepared by proceeding as in the following Reaction Scheme 1 below.

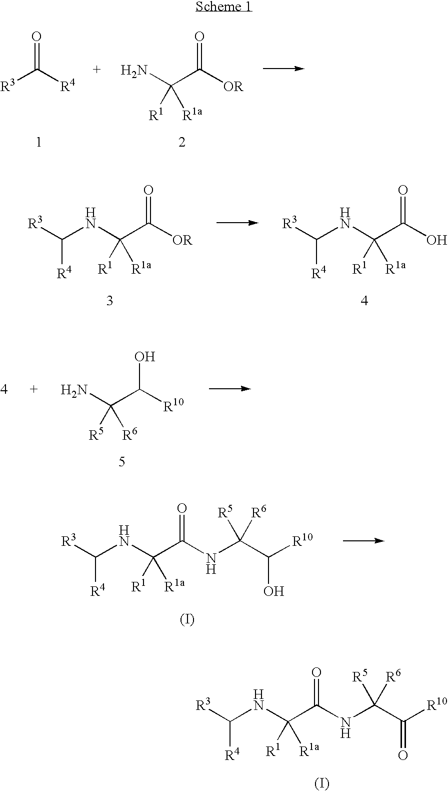

Reaction of a ketone of formula 1 where R³ is as defined in the Summary of the Invention and R⁴ is a haloalkyl (preferably trifluoromethyl) with an α-amino ester of formula 2 (where R is an alkyl group, preferably methyl, and R¹ and R^{1a} are as defined in the Summary of the Invention) under reductive amination reaction conditions provides a compound of formula 3. The reaction is carried out in the presence of a suitable dehydrating agent such as TiCl4, magnesium sulfate, isopropyl trifluoroacetate, in the presence of a base such as diisopropylethylamine, pyridine, and the like, and in a suitable organic solvent such as methylene chloride to give an imine. The imine is reduced with a suitable reducing agent such as sodium borohydride, sodium cyanoborohydride, and the like in a suitable organic solvent such as methanol, ethanol, and the like.

Compounds of formula 1 such as 2,2,2-trifluoromethylacetophenone are commercially available. Others can be prepared by methods well known in the art. α-Amino esters of formula 2 of alanine, cysteine, aspartic acid, glutamic acid, phenylalanine, histidine, and lysine are commercially available. Others can be prepared by methods well known in the art. Some such methods are described in PCT Applications Publication Nos. WO 03075836, WO 00/55144, WO 01/19816, WO 02/20485, WO 03/029200, U.S. Provisional Application No. 60/422,337, U.S. Pat. No. 6,353,017B1, 6,492,662B1, 353,017 B1 and 6,525,036B1, 6,229,011B1, 6,610,700, the disclosures of which are incorporated herein by reference in their entirety.

Hydrolysis of the ester group under aqueous basic hydrolysis reaction conditions provides the corresponding acid 4. The reaction is typically carried out with cesium carbonate, lithium hydroxide, and the like in an aqueous alcohol such as methanol, ethanol, and the like.

Alternatively, compounds of formula 4 can be prepared as shown in Method (i) below. Method (i):

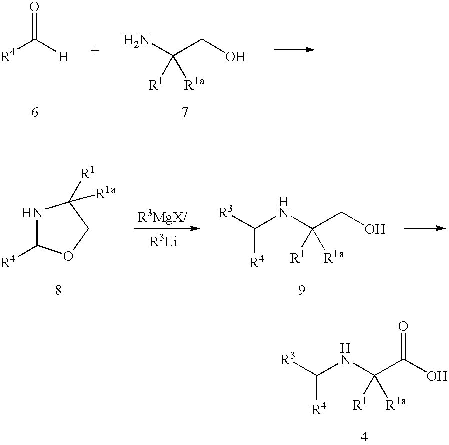

Condensation of an aldehyde of formula 6 with an aminoethanol of formula 7 utilizing Dean Stark apparatus provides a cyclic aminal 8 which upon reaction with a Grignard reagent of formula R³MgX (where X is halo) or an organolithium reagent R³Li provides a compound of formula 9. Oxidation of 9 with Jones oxidizing reagent or H₅IO₆/CrO₃ then provides compound 4.

Compound 9 can also be prepared by reacting O-protected aminoethanol 7 with a hemiacetal compound of formula R⁴C(OH)(OMe) to give an imine. Reaction of the imine with R³MgX or R³Li, followed by removal of the O-protecting group then provides 9.

Alternatively, a compound of formula 4 can be prepared shown in Method (ii) below. Method (ii):

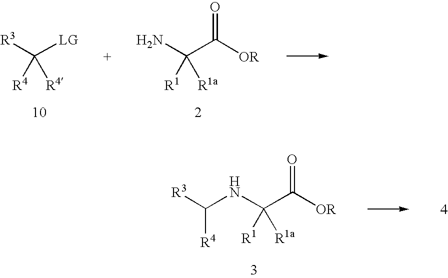

Reaction of a compound of formula 10 where LG is a suitable leaving group such as trifluoromethansulfonate, and the like, and R³, R⁴, and R^{4′} are as defined in Summary of the Invention with a compound of formula 2 where R¹, R^{1a} and R are as defined in the Summary of the Invention provides a compound of formula 3. The reaction is carried out in a suitable organic solvent, including but not limited to, halogenated organic solvents such as methylene chloride, 1,2-dibromoethane, and the like, ethereal solvents such as diethyl ether, tetrahydrofuran, acetonitrile, or aromatic solvents such as benzene, toluene, xylene, and the like, or mixtures thereof and optionally in the presence of an organic or inorganic base. Preferably, the organic base is triethylamine, pyridine, N-methylmorpholine, collidine, diisopropylethylamine, and the like. Preferably, the inorganic base is cesium carbonate, sodium carbonate, sodium bicarbonate, and the like. The reaction is optionally carried out in the presence of a drying agent such as molecular sieves. Preferably, the reaction is carried out at room temperature.

Compounds of formula 10 can be prepared by methods well known in the art. For example, a compound of formula 10 where R³ is phenyl or 4-fluorophenyl, R⁴ is trifluoromethyl, and R⁴ is hydrogen can be readily prepared from commercially available 2,2,2-trifluoroacetophenone or 2,2,2,4′-tetrafluoroacetophone respectively, by reducing the keto group to an alcoholic group by suitable reducing agent such as sodium borohydride, lithium aluminum hydride, and the like. The solvent used depends on the type of reducing agent. For example, when sodium borohydride is used the reaction is carried out in an alcoholic organic solvent such as methanol, ethanol, and the like. When lithium aluminum hydride is used the reaction is carried out in an ethereal solvent such as tetrahydrofuran, and the like. Reaction of 2,2,2-trifluoro-1-phenylethanol or 2,2,2-trifluoro-1-(4-fluorophenyl)ethanol with triflic anhydride provides the desired compound. Chirally enriched compound of formula 10 can be obtained by reduction of the corresponding halogenated acetophenone with a suitable reducing agent such as catecholborane or BH₃-DMS complex in the presence of a suitable catalyst such as (S) or (R)-CBS catalyst or (S) or (R)-α,α-diphenyl-2-pyrrolidine-methanol in the presence of BBN.

Compound 3 is then converted to a compound of formula 4 as described above.

Compound 4 is reacted with an α-aminoalcohol compound of formula 5 to provide a compound of Formula (I). The reaction is typically carried out in the presence of a suitable coupling agent e.g., benzotriazole-1-yloxytrispyrrolidinophosphonium hexafluorophosphate (PyBOP®), O-benzotriazol-1-yl-N,N,N′,N′-tetramethyl-uronium hexafluorophosphate (HBTU), O-(7-azabenzotriazol-1-yl)-1,1,3,3-tetramethyl-uronium hexafluorophosphate (HATU), 1-(3-dimethylaminopropyl)-3-ethylcarbodiimide hydrochloride (EDC), or 1,3-dicyclohexyl-carbodiimide (DCC), optionally in the presence of 1-hydroxybenzotriazole (HOBT), and a base such as N,N-diisopropylethylamine, triethylamine, N-methylmorpholine, and the like. The reaction is typically carried out at 20 to 30° C., preferably at about 25° C., and requires 2 to 24 h to complete. Suitable reaction solvents are inert organic solvents such as halogenated organic solvents (e.g., methylene chloride, chloroform, and the like), acetonitrile, N,N-dimethylformamide, ethereal solvents such as tetrahydrofuran, dioxane, and the like. Preferably, the reaction is carried out with HOBt, and EDC in dichloromethane.

Alternatively, compound (I) can be prepared from 4 by first converting 4 into an active acid derivative such as succinimide ester and then reacting it with an α-aminoalcohol 5. The conditions utilized in this reaction depend on the nature of the active acid derivative. For example, if it is an acid chloride derivative of 4, the reaction is carried out in the presence of a suitable base (e.g. triethylamine, diisopropylethylamine, pyridine, and the like). Suitable reaction solvents are polar organic solvents such as acetonitrile, N,N-dimethylformamide, dichloromethane, or any suitable mixtures thereof.

Compounds of formula 5 can be prepared under deprotonation reaction conditions by treating benzoxazole or oxazolo[4,5-b]pyridine, and the like, with a Grignard reagent such as isopropylmagnesium chloride and then reacting the resulting organomagnesium reagent with an alpha-(N-protected amino)aldehyde of formula R⁵R⁶C(NHPG)CHO, where R⁵ and R⁶ are as defined in the Summary of the Invention and PG is a suitable amino protecting group (such as tert-butyoxycarbonyl, benzyloxycarbonyl, or benzyl) to provide an N-protected compound of formula 5 after treatment with an aqueous acid or buffer. Removal of the amino protecting group then provides a compound of formula 5.

The addition reaction is typically carried out in an ethereal organic solvent such as tetrahydrofuran, diethyl ether, dioxane, and the like, preferably tetrahydrofuran, at a temperature from about −78° C. to about 40° C. Preferably, the reaction is carried out from about −10° C. to about 40° C., more preferably from about −10° C. to about 10° C. The reaction typically requires an hour to complete. The nucleophilic addition reaction is typically carried out from about −10° C. to about room temperature. Compounds of formula R⁵R⁶C(NHPG)CHO are prepared from commercially available starting materials by methods well known in the art.

The reaction conditions employed for removal of the amino protecting group depends on the nature of the protecting group. For example, if the protecting group is tert-butoxycarbonyl, it is removed under acid reaction conditions. Suitable acids are trifluoroacetic acid (TFA), hydrochloric acid, and the like. If the protecting group is benzyl or benzyloxycarbonyl, it is removed under catalytic hydrogenation reaction conditions. Suitable catalyst are palladium, platinum, rodium based catalysts and others known in the art. Other suitable reaction conditions for their removal can be found in Greene, T. W.; and Wuts, P. G. M.; Protecting Groups in Organic Synthesis; John Wiley & Sons, Inc. 1999. The reaction is carried out in an inert organic solvent methylene chloride, tetrahydrofuran, dioxane, dimethylformamide, and the like.

It will be apparent to a person skilled in the art, that compounds of Formula (I) can also be prepared under the reaction conditions described above, by first condensing 5 with the N-protected amino acid of formula 2 where R is hydrogen followed by removal of the amino protecting group and reacting the free amino compound with a compound of formula 1.

A compound of Formula (I) can be converted to other compounds of Formula (I). For example, as shown in Scheme 1 above, oxidation of hydroxy group in (I) i.e., compound (I) where R⁷ is hydroxy and R⁸ is hydrogen provides a corresponding compound of Formula I where R⁷ and R⁸ together from oxo. The reaction is carried out with a suitable oxidizing agent such as Dess-Martin Periodinane in a halogenated organic solvent such as methylene chloride, chloroform, carbon tetrachloride, and the like, or a mixture of TEMPO/bleach.

Additionally, the above procedure can also be used to prepared compounds of Formula (I) where R^{4′} is other than hydrogen utilizing the procedure described in method (i) above, by substituting R^{4′}COH with R⁴R^{4′}CO and then treating the resulting cyclic aminal with R³Li/R³MgX, followed by oxidation to give the free acid. The free acid is then condensed with under conditions described above to give compound (I).

Alternatively, compounds of Formula (I) where E is —C(R⁵)(R⁶)C(R⁷)(R⁸)R¹⁰ and where R¹, R^{1a}, R², R³, R⁴, R⁵, R⁶, R⁷ and R⁸ are as defined in the Summary of the Invention and R^{4′} is hydrogen can be prepared by proceeding as in the following Reaction Scheme 2 below.

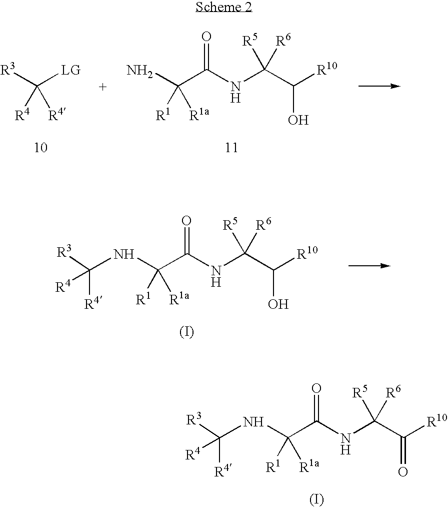

Reaction of a compound of formula 10 where LG is a suitable leaving group such as trifluoromethansulfonate, and the like, and R³, R⁴, and R^{4′} are as defined in Summary of the Invention with a compound of formula 11 where R¹, R^{1a}, R⁵, R⁶, and R¹⁰ are as defined in the Summary of the Invention, R⁷ is hydrogen and R⁸ is hydroxy provides a compound of Formula (I) where R¹, R^{1a}, R³, R⁴, R^{4′}, R⁵, R⁶, and R¹⁰ are as defined in the Summary of the Invention, R⁷ is hydrogen and R⁸ is hydroxy. The reaction is carried out as described above.

Compounds of formula 11 can be prepared by methods well known in the art. A compound of Formula (I) where which can then be converted to a corresponding compound of Formula (I) where R⁷ is hydrogen and R⁸ is hydroxy is converted to a corresponding compound of Formula (I) where R⁷ and R⁸ are oxo as described in Scheme 1 above

Compounds of Formula (I) where E is —C(R⁵)(R⁶)CH═CHS(O)₂R¹⁰ can be prepared as shown in Scheme 3 below.

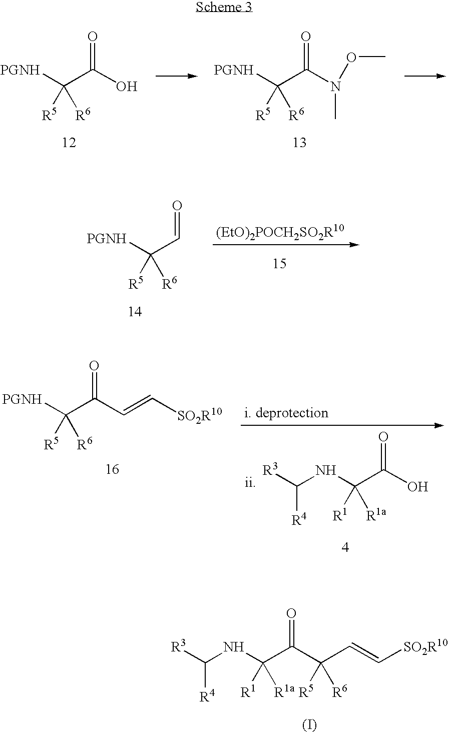

Reaction of an N-protected amino acid of formula 12 with N,O-methylhydroxylamine hydrochloride in the presence of 1 equivalent of triethylamine and N)N-dicyclohexylcarbodiimide forms the N,O-dimethylhydroxamate (Weinreb amide) 13, which is then reduced to the corresponding aldehyde 14 with a suitable reducing agent such as 0.5 equivalents of lithium aluminum hydride.

Condensation of 14 with a Wadsworth-Emmons reagent (EtO)₂POCH₂SO₂R¹⁰ 15, wherein R¹⁰ is as defined in the Summary of the Invention, affords the vinyl sulfone 16. Removal of the amino protecting group, following by reaction of the resulting free amine with a compound of formula 4 as described above then provides a compound of Formula (I).

Other compounds of Formula (I) can be prepared by methods disclosed in US and PCT Applications publication Nos. 2003/0092634A1, WO 02/098850 and WO 03/024924, U.S. Pat. Nos. 6,506,733 the disclosures of which are incorporated herein by referenced in their entirety.

Additional Processes for Preparing Compounds of Formula (I):

A compound of Formula (I) can be prepared as a pharmaceutically acceptable acid addition salt by reacting the free base form of the compound with a pharmaceutically acceptable inorganic or organic acid. Alternatively, a pharmaceutically acceptable base addition salt of a compound of Formula (I) can be prepared by reacting the free acid form of the compound with a pharmaceutically acceptable inorganic or organic base. Inorganic and organic acids and bases suitable for the preparation of the pharmaceutically acceptable salts of compounds of Formula (I) are set forth in the definitions section of this Application. Alternatively, the salt forms of the compounds of Formula (I) can be prepared using salts of the starting materials or intermediates.

The free acid or free base forms of the compounds of Formula (I) can be prepared from the corresponding base addition salt or acid addition salt form. For example, a compound of Formula (I) in an acid addition salt form can be converted to the corresponding free base by treating with a suitable base (e.g., ammonium hydroxide solution, sodium hydroxide, and the like). A compound of Formula (I) in a base addition salt form can be converted to the corresponding free acid by treating with a suitable acid (e.g., hydrochloric acid, etc).

The N-oxides of compounds of Formula (I) can be prepared by methods known to those of ordinary skill in the art. For example, N-oxides can be prepared by treating an unoxidized form of the compound of Formula (I) with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds of Formula (I) can be prepared from the N-oxide of an appropriate starting material.

Compounds of Formula (I) in unoxidized form can be prepared from N-oxides of compounds of Formula (I) by treating with a reducing agent (e.g., sulfur, sulfur dioxide, triphenyl phosphine, lithium borohydride, sodium borohydride, phosphorus trichloride, tribromide, or the like) in an suitable inert organic solvent (e.g., acetonitrile, ethanol, aqueous dioxane, or the like) at 0 to 80° C.

Prodrug derivatives of the compounds of Formula a) can be prepared by methods known to those of ordinary skill in the art (e.g., for further details see Saulnier et al. (1994), Bioorganic and Medicinal Chemistry Letters, Vol. 4, p. 1985). For example, appropriate prodrugs can be prepared by reacting a non-derivatized compound of Formula (I) with a suitable carbamylating agent (e.g., 1,1-acyloxyalkylcarbonochloridate, para-nitrophenyl carbonate, or the like).

Protected derivatives of the compounds of Formula (I) can be made by means known to those of ordinary skill in the art. A detailed description of the techniques applicable to the creation of protecting groups and their removal can be found in T. W. Greene, Protecting Groups in Organic Synthesis, 3^{rd} edition, John Wiley & Sons, Inc. 1999.

Compounds of the present invention may be conveniently prepared, or formed during the process of the invention, as solvates (e.g. hydrates). Hydrates of compounds of the present invention may be conveniently prepared by recrystallisation from an aqueous/organic solvent mixture, using organic solvents such as dioxin, tetrahydrofuran or methanol.

Compounds of Formula (I) can be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diasteromeric derivatives of compounds of Formula (I), dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

Preparation of Biological Agents

In practicing this invention several processes for the generation or purification of biological agents are used. Methods for preparing the biologics are well known in the art as discussed below.

Monoclonal antibodies are prepared using standard techniques, well known in the art, such as by the method of Kohler and Milstein, Nature 1975, 256:495, or a modification thereof, such as described by Buck et al. 1982, In Vitro 18:377. Typically, a mouse or rat is immunized with the MenB PS derivative conjugated to a protein carrier, boosted and the spleen (and optionally several large lymph nodes) removed and dissociated into single cells. If desired, the spleen cells may be screened (after removal of non-specifically adherent cells) by applying a cell suspension to a plate or well coated with the antigen. B-cells, expressing membrane-bound immunoglobulin specific for the antigen, will bind to the plate, and will not be rinsed away with the rest of the suspension. Resulting B-cells, or all dissociated spleen cells, are then induced to fuse with myeloma cells to form hybridomas. Representative murine myeloma lines for use in the hybridizations include those available from the American Type Culture Collection (ATCC).

Chimeric antibodies composed of human and non-human amino acid sequences may be formed from the mouse monoclonal antibody molecules to reduce their immunogenicity in humans (Winter et al. Nature 1991 349:293; Lobuglio et al. Proc. Nat. Acad. Sci. USA 1989 86:4220; Shaw et al. J. Immunol. 1987 138:4534; and Brown et al. Cancer Res. 1987 47:3577; Riechmann et al. Nature 1988 332:323; Verhoeyen et al. Science 1988 239:1534; and Jones et al. Nature 1986 321:522; EP Publication No. 519,596, published Dec. 23, 1992; and U.K. Patent Publication No. GB 2,276,169, published Sep. 21, 1994).

Antibody molecule fragments, e.g., F(ab′).sub.2, FV, and sFv molecules, that are capable of exhibiting immunological binding properties of the parent monoclonal antibody molecule can be produced using known techniques. Inbar et al. Proc. Nat. Acad. Sci. USA 1972 69:2659; Hochman et al. Biochem. 1976 15:2706; Ehrlich et al. Biochem. 1980 19:4091; Huston et al. Proc. Nat. Acad. Sci. USA 1988 85(16):5879; and U.S. Pat. Nos. 5,091,513 and 5,132,405, and U.S. Pat. No. 4,946,778.

In the alternative, a phage-display system can be used to expand the monoclonal antibody molecule populations in vitro. Saiki, et al. Nature 1986 324:163; Scharf et al. Science 1986 233:1076; U.S. Pat. Nos. 4,683,195 and 4,683,202; Yang et al. J. Mol. Biol. 1995 254:392; Barbas, III et al. Methods: Comp. Meth Enzymol. 1995 8:94; Barbas, III et al. Proc. Natl. Acad. Sci. USA 1991 88:7978.

The coding sequences for the heavy and light chain portions of the Fab molecules selected from the phage display library can be isolated or synthesized, and cloned into any suitable vector or replicon for expression. Any suitable expression system can be used, including, for example, bacterial, yeast, insect, amphibian and mammalian systems. Expression systems in bacteria include those described in Chang et al. Nature 1978 275:615, Goeddel et al. Nature 1979 281:544, Goeddel et al. Nucleic Acids Res. 1980 8:4057, European Application No. EP 36,776, U.S. Pat. No. 4,551,433, deBoer et al. Proc. Natl. Acad. Sci. USA 1983 80:21-25, and Siebenlist et al. Cell 1980 20:269.

Expression systems in yeast include those described in Hinnen et al. Proc. Natl. Acad. Sci. USA 1978 75:1929, Ito et al. J. Bacteriol. 1983 153:163, Kurtz: et al. Mol. Cell. Biol. 1986 6:142, Kunze et al. J. Basic Microbiol. 1985 25:141, Gleeson et al. J. Gen. Microbiol. 1986 132:3459, Roggenkamp et al. Mol. Gen. Genet. 1986 202:302, Das et al. J. Bacteriol. 1984 158:1165, De Louvencourt et al. J. Bacteriol. 1983 154:737, Van den Berg et al. Bio/Technology 1990 8:135, Kunze et al. J. Basic Microbiol. 1985 25:141, Cregg et al. Mol. Cell. Biol. 1985 5:3376, U.S. Pat. Nos. 4,837,148 and 4,929,555, Beach et al. Nature 1981 300:706, Davidow et al. Curr. Genet. 1985 10:380, Gaillardin et al. Curr. Genet. 1985 10:49, Ballance et al. Biochem. Biophys. Res. Commun. 1983 112:284-289, Tilburn et al. Gene 1983 26:205-221, Yelton et al. Proc. Natl. Acad. Sci. USA 1984 81:1470-1474, Kelly et al. EMBO J. 1985 4:475479; European Application No. EP 244,234, and International Publication No. WO 91/00357.

Expression of heterologous genes in insects can be accomplished as described in U.S. Pat. No. 4,745,051, European Application Nos. EP 127,839 and EP 155,476, Vlak et al. J. Gen. Virol. 1988 69:765-776, Miller et al. Ann. Rev. Microbiol. 1988 42:177, Carbonell et al. Gene 1988 73:409, Maeda et al. Nature 1985 315:592-594, Lebacq-Verheyden et al. Mol. Cell. Biol. 1988 8:3129, Smith et al. Proc. Natl. Acad. Sci. USA 1985 82:8404, Miyajima et al. Gene 1987 58:273, and Martin et al. DNA 1988 7:99. Numerous baculoviral strains and variants and corresponding permissive insect host cells from hosts are described in Luckow et al. Bio/Technology 1988 6:47-55, Miller et al. GENETIC ENGINEERING, Setlow, J. K. et al. eds., Vol. 8, Plenum Publishing, pp. 1986 277-279, and Maeda et al. Nature 1985 315:592-594.

Mammalian expression can be accomplished as described in Dijkema et al. EMBO J. 1985 4:761, Gorman et al. Proc. Natl. Acad. Sci. USA 1982 79:6777, Boshart et al. Cell 1985 41:521, and U.S. Pat. No. 4,399,216. Other features of mammalian expression can be facilitated as described in Ham et al. Meth. Enz. 1979 58:44, Barnes et al. Anal. Biochem. 1980 102:255, U.S. Pat. Nos. 4,767,704, 4,657,866, 4,927,762, 4,560,655 and Reissued U.S. Pat. No. RE 30,985, and in International Publication Nos. WO 90/103430, WO 87/00195.

The production of recombinant adenoviral vectors are described in U.S. Pat. No. 6,485,958.

Botulinum toxin type A can be obtained by establishing and growing cultures of Clostridium botulinum in a fermenter and then harvesting and purifying the fermented mixture in accordance with known procedures.

Any of the above-described protein production methods can be used to provide the biologic that would benefit from the present invention.

### Utility

The compounds of the invention are selective inhibitors of cysteine proteases, in particular, cathepsin S, K, B, and/or F, and accordingly are useful for treating diseases in which cysteine protease activity contributes to the pathology and/or symptomatology of the disease. For example, the compounds of the invention are useful in treating autoimmune disorders, including, but not limited to, juvenile onset diabetes, psoriasis, multiple sclerosis, pemphigus vulgaris, Graves' disease, myasthenia gravis, systemic lupus erythemotasus, rheumatoid arthritis and Hashimoto's thyroiditis, allergic disorders, including, but not limited to, asthma, allogeneic immune responses, including, but not limited to, organ transplants or tissue grafts and endometriosis.

Cathepsin S is also implicated in disorders involving excessive elastolysis, such as chronic obstructive pulmonary disease (e.g., emphysema), bronchiolitis, excessive airway elastolysis in asthma and bronchitis, pneumonities and cardiovascular disease such as plaque rupture and atheroma. Cathepsin S is implicated in fibril formation and, therefore, inhibitors of cathepsins S are of use in treatment of systemic amyloidosis. Additionally, the intermediates 4 are useful as α4 integrins, such as VLA4 and α4β7, antagonists and are therefore useful for treating diseases such as chronic inflammatory disease such a rheumatoid arthritis, multiple sclerosis, asthma, inflammatory bowel disease, and Crohn's diseases.

### Testing

The cysteine protease inhibitory activities of the compounds of Formula (I) can be determined by methods known to those of ordinary skill in the art. Suitable in vitro assays for measuring protease activity and the inhibition thereof by test compounds are known. Typically, the assay measures protease-induced hydrolysis of a peptide-based substrate. Details of assays for measuring protease inhibitory activity are set forth in Biological Examples 1-5, infra.

The VLA-4 antagonist activity of intermediate 4 can be tested by utilizing the assays described in U.S. Pat. Nos. 6,229,011 and 6,482,840 the disclosures of which are incorporated herein by reference in their entirety.

### Administration and Pharmaceutical Compositions

In general, compounds of Formula (I) will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. For example, therapeutically effective amounts of a compound of Formula (I) may range from about 10 micrograms per kilogram body weight (μg/kg) per day to about 20 milligram per kilogram body weight (mg/kg) per day, typically from about 100 μg/kg/day to about 10 mg/kg/day. Therefore, a therapeutically effective amount for an 80 kg human patient may range from about 1 mg/day to about 1.6 g/day, typically from about 1 mg/day to about 100 mg/day. In general, one of ordinary skill in the art, acting in reliance upon personal knowledge and the disclosure of this Application, will be able to ascertain a therapeutically effective amount of a compound of Formula (I) for treating a given disease.

The compounds of Formula (I) can be administered as pharmaceutical compositions by one of the following routes: oral, systemic (e.g., transdermal, intranasal or by suppository) or parenteral (e.g., intramuscular, intravenous or subcutaneous). Compositions can take the form of tablets, pills, capsules, semisolids, powders, sustained release formulations, solutions, suspensions, elixirs, aerosols, or any other appropriate composition and are comprised of, in general, a compound of Formula (I) in combination with at least one pharmaceutically acceptable excipient. Acceptable excipients are non-toxic, aid administration, and do not adversely affect the therapeutic benefit of the active ingredient. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from water, ethanol, glycerol, propylene glycol and various oils, including those of petroleum, animal, vegetable or synthetic origin (e.g., peanut oil, soybean oil, mineral oil, sesame oil, and the like). Preferred liquid carriers, particularly for injectable solutions, include water, saline, aqueous dextrose and glycols.

The amount of a compound of Formula (I) in the composition may vary widely depending upon the type of formulation, size of a unit dosage, kind of excipients and other factors known to those of skill in the art of pharmaceutical sciences. In general, a composition of a compound of Formula (I) for treating a given disease will comprise from 0.01% w to 10% w, preferably 0.3% w to 1% w, of active ingredient with the remainder being the excipient or excipients. Preferably the pharmaceutical composition is administered in a single unit dosage form for continuous treatment or in a single unit dosage form ad libitum when relief of symptoms is specifically required. Representative pharmaceutical formulations containing a compound of Formula (I) are described in Example 1 below.

### EXAMPLES

The present invention is further exemplified, but not limited by, the following examples that illustrate the preparation of compounds of Formula (I) (Examples) and intermediates (References) according to the invention.

### Example A

### Synthesis of 2(RS)-benzyloxycarbonylamino-4(RS)-(2-methoxyphenyl)pentanoic acid

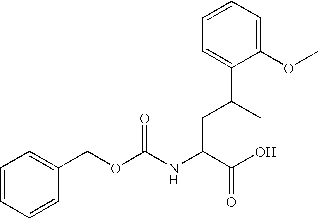

To d,l-2-methoxy-α-methylbenzyl alcohol (0.5 g, 3.29 mmol) was added 48% aq. HBr (2 mL) and the reaction mixture was stirred rapidly for 1.5 h. The reaction mixture was diluted with hexane (30 mL), washed with water, dried with MgSO₄, filtered, and evaporated under vacuum. The crude d,l-2-methoxy-α-methylbenzyl bromide was added to a solution of tributyltin hydride (0.67 mL, 2.49 mmol), Z-dehydroalanine methyl ester (0.25 g, 1.06 mmol), and 2,2′-azobisisobutyronitrile (15 mg, 0.09 mmol) in benzene (5 mL). The reaction mixture was heated at 80° C. under a nitrogen atmosphere for 5 h. Benzene was removed under vacuum and the residue was dissolved in methanol (20 mL). 2N KOH (5 mL) was added and the mixture was rapidly stirred at room temperature over night. Methanol was removed under vacuum and the residue was diluted with water (20 mL). The aqueous solution was washed with ether to remove the tin by products. The aqueous layer was acidified with 6 N HCl (aq.) and the product was extracted with ethyl acetate. The combined organic layers were washed with brine, dried with MgSO₄, filtered, and evaporated under vacuum to give 2-benzyloxy-carbonylamino-4-(2-methoxyphenyl)pentanoic acid (190 mg, 0.53 mmol) as a mixture of diastereomers in sufficiently pure form to be used without further purification. MS: (M⁺+H) 358, (M⁺−H) 356.

Following the procedure described above, and utilizing appropriate starting materials the following amino acids were prepared:
2-benzyloxycarbonylamino-4-(2-methoxyphenyl)hexanoic acid;
2-benzyloxy-carbonylamino-4-(4-fluorophenyl)pentanoic acid;
2-benzyloxy-carbonylamino-4-(4-chlorophenyl)pentanoic acid;
2-benzyloxy-carbonylamino-4-(4-methoxyphenyl)pentanoic acid;
2-benzyloxy-carbonylamino-4-(2-trifluoromethylphenyl)pentanoic acid;
2-benzyloxy-carbonylamino-4-(3-trifluoromethylphenyl)pentanoic acid;
2-benzyloxy-carbonylamino-4-(napth-1-yl)pentanoic acid;
2-benzyloxy-carbonylamino-4-(2,6-dimethylphenyl)pentanoic acid;
2-benzyloxy-carbonylamino-4-(2,4-difluorophenyl)pentanoic acid;
2-benzyloxy-carbonylamino-4-(2,4-dimethylphenyl)pentanoic acid;
2-benzyloxy-carbonylamino-4-(2,5-dimethylphenyl)pentanoic acid; and
2-benzyloxy-carbonylamino-4-(2,4-dichlorophenyl)pentanoic acid.

The benzyloxycarbonyl group can be removed as described in Example C below to give the corresponding free amino acid.

### Example B

### Synthesis of 2(S)-2,6-difluorophenylalanine

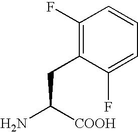 Step 1

N-(Benzyloxycarbonyl)-α-phosphonoglycine trimethyl ester (Aldrich No. 37,635-3; 6.7 g, 20 mmol) and 1,8-diazabicyclo[5,4,0]undec-7-ene (Aldrich No. 13, 900-9; 3.3 mL, 22 mmol) were dissolved in methylene chloride (11 mL) and stirred at room temperature for 15 min., and then cooled to <−30° C. A solution of 2,6-difluorobenzaldehyde (1.9 mL, 20 mmol) in methylene chloride (25 mL) was added to the reaction mixture dropwise over 20 min. The reaction mixture was stirred for another 20 min., and then allowed to warm up to room temperature for 30 min. The reaction mixture was then poured into ethyl ether (300 mL) and washed with 1 N HCl, brine and dried over MgSO₄. Rotary evaporation gave 2-benzyloxycarbonylamino-3-(2,6-difluorophenyl)acrylic acid methyl ester. This crude product was purified by chromatography on a Medium Pressure Liquid Column (MPLC) eluting with 20% ethyl acetate/80% hexane to give pure product (5 g, 72% yield, liquid).

Step 2

A mixture of 2-benzyloxycarbonylamino-3-(2,6-difluorophenyl)acrylic acid methyl ester (14.4 mmol), and catalyst, (+)-1,2-bis-[(2S,5S)2,5-diethylphopholano]benzene (cyclooctadiene)rhodium (I) trifluoromethanesulfonate (Strem. Chemical No. 45-0151; 104 mg, 0.14 mmol) was dissolved in ethanol (150 mL). Hydrogenation was performed at 50 psi H₂ at room temperature over 2 days. The solvent was then removed by rotary evaporation to give 2(S)-benzyloxycarbonylamino-3-(2,6-difluorophenyl)propionic acid methyl ester.

Step 3

2(S)-Benzyloxycarbonylamino-3-(2,6-difluorophenyl)propionic acid methyl ester (5 g, 14.4 mmol) was dissolved in methanol (60 mL) and cooled on ice. 1 N NaOH (22 mL, 22 mmol) was added dropwise over 15 min. The reaction mixture was removed from cooling and continue stirring at room temperature for 4 h. The solvent was then removed by rotary evaporation. The residue was treated with water (100 mL) and then with 1 N HCl to adjust the pH to 4. The product was extracted with ethyl acetate (300 mL, 200 mL). Evaporation of the solvent and crystallization of the residue from methylene chloride/hexane gave 2(S)-benzyloxycarbonylamino-3-(2,6-difluoro-phenyl)propionic acid (4.6 g, 13.7 mmol, 94% yield).

Step 4

2(S)-Benzyloxycarbonylamino-3-(2,6-difluorophenyl)-propionic acid was hydrogenated at 50 psi in ethanol (25 mL) in the presence of 5% palladium on activated carbon (600 mg) for 24 h. The catalyst was removed by filtration through celite and the solvent evaporated to give a residue which was crystallized from ethyl ether to give 2(S)-2,6-difluorophenylalanine (2.2 g, 11 mmol, 80% yield). ¹H NMR (DMSO-d₆): δ 7.28 (m, 1H), 7.0 (t, J=7.6 Hz, 2H), 2.77 (m, 2H). MS: 202.2 (M+1), 199.7 (M−1).

### Example C

### Synthesis of 2(RS)-amino-4(RS)-6,6-trimethylheptanoic acid

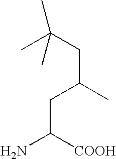 Step 1

To a mixture of the 3,5,5-trimethylhexanal (17.4 mL, 0.10 mol), ammonium chloride (53.5 g, 0.205 mol) and diethyl ether (113 mL) was added sodium cyamide (7.35 g, 0.15 mol) in water (38 mL). The reaction mixture was allowed to stir vigorously for 16 h. The layers were separated. The aqueous layer was extracted with diethyl ether. The combined organic layer was then extracted with 1 N HCl. Saturated sodium bicarbonate was then added until 1-cyano-3,5,5-trimethyl-hexylamine was completely precipitated. Vacuum filtration and washing with 5 mL ice cold water followed by lyophilization gave 1-cyano-3,5,5-trimethylhexylamine (5.805 g, 0.034 mol, 34.5%) as a white solid.

Step 2

1-Cyano-3,5,5-trimethylhexylamine (1.02 g, 5.0 mmol) was treated with 6 N HCl (10 mL) and heated at reflux for 30 h. The reaction mixture was allowed to cool to room temperature. Water (50 mL) was added, and the mixture was washed with diethyl ether. The aqueous layer was basified to pH 8.5 with 2 M KOH. A white precipitate formed which was collected by vacuum filtration and lyophilized to give 2(RS)-amino-4(RS),6,6-trimethyl-heptanoic acid (364 mg).

### Example D

### Synthesis of 2(RS)-amino-4-methyl-4-phenylpentanoic acid

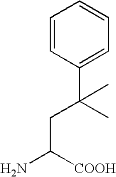 Step 1

4-Methyl-4-phenyl-1-pentene was prepared by reacting 2-phenyl-2-propanol with 3-(trimethylsilyl)propene by the method of Cella, J. Org. Chem., 1982, 47, 2125-2130.

Step 2

4-Methyl-4-phenyl-1-pentene was ozonolyzed at −78° C. in dichloromethane followed by dimethyl sulfide quenching to give crude product which was purified by silica gel chromatography to give 3-methyl-3-phenylbutanal which was then converted to the title compound by proceeding as described in PCT application publication No. WO 2004/052921, Reference C, on page 68 of the application.

### Example E

### Synthesis of 2(RS)-benzyloxycarbonylamino-4-ethylhexanoic acid

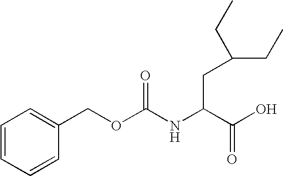 Step 1

A mixture of 2-benzyloxycarbonylaminomalonic acid diethyl ester (Bladon, C. M. J. Chem. Soc. Perkin Trans. 1990, 1, 1151-1158) (1.237 g), iodo-2-ethylbutane (1.272 g) and lithium hydroxide (0.287 g) in N-methylpyrrolidone (8 mL) was stirred for 2 days at room temperature and then diluted with ice water. The aqueous solution was extracted with ether and the product purified by chromatography on silica gel to give 2-benzyloxycarbonylamino-2-(2-ethylbutyl)malonic acid diethyl ester (0.520 g).

Step 2

A solution of 2-benzyloxycarbonylamino-2-(2-ethylbutyl)malonic acid diethyl ester (0.520 g) in ethanol (5 mL) was treated with sodium hydroxide (2.91 mL, 1 N) and then stirred at room temperature for 8 h. The reaction mixture was diluted with water and acidified with HCl and the product was then extracted with ethyl acetate to give 2-benzyloxycarbonylamino-2-(2-ethylbutyl)malonic acid monoethyl ester (0.461 g).

Step 3

2-Benzyloxycarbonylamino-2-(2-ethylbutyl)malonic acid monoethyl ester was heated at 75° C. in ethanol (5 mL) with sodium hydroxide (5 mL, 1 N) for 3 h and 2-benzyloxycarbonyl-amino-2-(2-ethylbutyl)malonic acid was isolated by extraction of the acidified reaction mixture. 2-Benzyloxycarbonylamino-2-(2-ethylbutyl)malonic acid was heated at 103° C. for 1 h and the resulting residue was purified by column chromatography on silica gel to give 2(RS)-benzyloxycarbonylamino-4-ethylhexanoic acid (0.220 g).

### Example F

### Synthesis of 2(S)-benzyloxycarbonylamino-3-pyrazol-1-ylpropionic acid

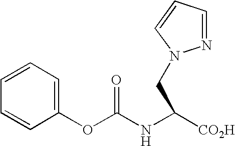

The title compound was prepared by treating S-benzyloxycarbonylserine-β-lactone with pyrazole in acetonitrile at 60° C. for 16 h (see J. Am. Chem. Soc., 1985, 107, 7105-7109).

Following the procedure described above, but substituting pyrazole with 1,2,4-triazole and 1,2,3-triazole provided 2(S)-benzyloxycarbonylamino-3-[1,2,4]-triazol-1-ylpropionic acid and 2(S)-benzyloxycarbonylamino-3-[1,2,3]-triazol-1-ylpropionic acid respectively.

### Example G

### Synthesis of 2(S)-tert-butoxycarbonyl)amino-1-(oxazolo[4,5-b]pyridin-2-yl)butan-1-ol

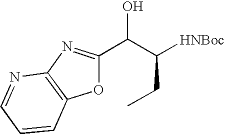 Step 1

A mixture of 2-amino-3-hydroxypyridine (11 g, 100 mmol), triethylorthoformate (80 mL) and p-toluenesulfonic acid (61 mg) was heated at 140° C. for 8 h. Excess triethylorthoformate was removed under vacuum and oxazolo[4,5-b]pyridine was crystallized from ethyl acetate (9 g).

Step 2

In a clean roundbottom flask equipped with stir bar was placed oxazolo[4,5-b]pyridine (600 mg, 5 mmol) in THF (30 mL) and the reaction mixture was cooled to 0° C. under N₂ atmosphere. Isopropylmagnesium chloride (2 M in THF, 2.5 mL, 5 mmol) was added. After stirring for 1 h at 0° C., (S)-2-(tert-butoxycarbonyl)aminobutyraldehyde (573 mg, 3 mmol) in THF (20 mL) was added. The ice bath was removed and the reaction mixture was allowed to warm to room temperature. After 2 h, the reaction mixture was quenched with saturated ammonium chloride solution and concentrated to dryness. The residue was extracted with EtOAc, then washed with brine, dried with anhyd. MgSO₄, filtered and concentrated. The crude product was purified by chromatograph to yield 383 mg of the desired compound.

H¹ NMR (DMSO-d₆): δ 8.42 (m, 1H), 8.18 (m, 1H), 7.3 (m, 1H), 6.8-6.6 (dd, d, 1H, OH, diastereomer), 6.3-6.02 (d, d, 1H, NH, diastereomer), 4.82-4.5 (m, m, 1H, diastereomer), 1.8-1.3 (m, 2H), 1.2-1.05 (s, s, 9H, diastereomer), 0.89 (m, 3H). MS: 306.2 (M−1), 308.6 (M+1).

### Example H

### Synthesis of 2(S)-(tert-butoxycarbonyl)amino-3-thiazol-2-ylpropionic acid

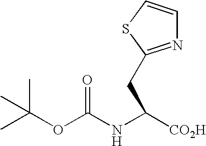

To 2-tert-butoxycarbonylamino-3-thiazol-2-yl-propionic acid methyl ester (500 mg, 1.75 mmol) in a mixture of acetonitrile (6 mL) and 0.2 M aqueous NaHCO₃ (12 mL) was added Alcalase (2.4 L, 0.08 mL), and the solution was stirred vigorously at room temperature for about 2.5 h. The reaction mixture was then evaporated at 30° C. to remove acetonitrile, and the aqueous residue was washed with ether. The aqueous phase was acidified with 6 N HCl to pH 3 and the solution was extracted with ethyl acetate. The combined organic layers were then dried and evaporated to yield 2(S)-tert-butoxycarbonyl-amino-3-thiazol-2-yl-propionic acid (204 mg).

### Reference I

### Synthesis of 4(S)-amino-2,2-difluoro-3-hydroxyhexanoic acid dimethylamide

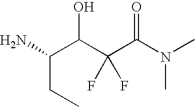

Activated zinc dust (2.16 g, 33 mmol) was suspended in dry THF (2 mL). A mixture of ethyl bromodifluoro acetate (6.5 g, 32 mmol) and (1S)-(1-formylpropyl) carbamic acid tert-butyl ester (2 g, 10.7 mmol), in THF (10 mL), was added over 20 min while the reaction mixture was sonicated. After complete addition, sonication was continued for a further 30 min. The reaction mixture was then diluted with ethyl acetate (200 mL) and washed with 1N aqueous KHSO₄, brine, dried with magnesium sulfate and evaporated. The crude product was dissolved in ethanol (15 mL) and a solution of dimethylamine (40% in water; 2 mL) was added. After stirring for 16 h at ambient temperature, the solvents were evaporated and the product was purified by flash chromatography on silica gel (hexane/ethyl acetate ratio of 3:1) to yield 200 mg 4(S)-Boc-amino-2,2-difluoro-3-hydroxy-hexanoic acid dimethylamide of colorless oil which was dissolved in a mixture of TFA/dichloromethane (1:1; 6 mL), stirred for 1 h and evaporated to dryness. The product, (S)-4-amino-2,2-difluoro-3-hydroxyhexanoic acid dimethylamide, was obtained as the TFA salt and used without further purification.

### Reference J

### Synthesis of 3(S)-amino-2-hydroxy-pentanoic acid benzylamide

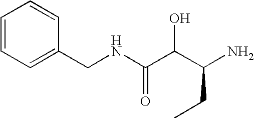 Step 1

(1S)-(2-Cyano-1-ethyl-2-hydroxyethyl)carbamic acid tert-butyl ester (10 g, 46.7 mmol) was dissolved in 1,4-dioxane (100 mL). Anisole (5 mL) was added and then concentrated HCl (100 mL). The reaction mixture was heated under reflux for 24 h. The reaction mixture was evaporated to dryness under vacuum and re-dissolved in 100 mL water. The solution was washed with ether and then neutralized with saturated aqueous NaHCO₃. Di-tert-butyl dicarbonate (10 g, 46 mmol) was added with 1,4-dioxane (200 mL), and the reaction mixture was stirred at ambient temperature for 24 h. The dioxane was removed under vacuum and the remaining aqueous solution was washed with ether. The solution was acidified with 1N HCl and extracted with ethyl acetate. The combined organic layers were washed with brine, dried with magnesium sulfate and evaporated to yield 3(S)-tert-butoxycarbonylamino-2-hydroxy-pentanoic acid (4.5 g) as yellowish oil.

Step 2

3(8)-tert-Butoxycarbonylamino-2-hydroxypentanoic acid (300 mg, 1.29 mmol) was combined with EDC (400 mg, 2.1 mmol) and HOBt (400 mg, 2.6 mmol). A solution of benzylamine (0.22 mL) and 4-methylmorpholine (0.5 mL) in dichloromethane (4 mL) was added in one portion. The reaction mixture was stirred at ambient temperature for 2 h. After dilution with ethyl acetate (150 mL), the solution was washed with 1 N aqueous HCl, water, saturated aqueous NaHCO₃ solution and brine. The resultant mixture was dried with magnesium sulfate and evaporated under vacuum to yield 3(S)-tert-butoxycarbonylamino-2-hydroxy-pentanoic acid benzylamide (380 mg) as a white solid.

Step 3

3(S)-tert-Butoxycarbonylamino-2-hydroxypentanoic acid benzylamide was dissolved in a mixture of TFA/dichloromethane (1:1; 6 mL), stirred for 1 h and evaporated to dryness. 3(S)-Amino-2-hydroxypentanoic acid benzylamide was obtained as the TFA salt and used without further purification.

### Reference K

### Synthesis of 2(S)-amino-1-(3-phenyl-[1,2,4]oxadiazol-5-yl)-butan-1-ol

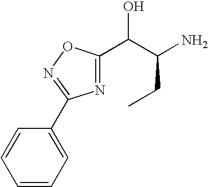

3(S)-tert-Butoxycarbonylamino-2-hydroxypentanoic acid (500 mg, 2.14 mmol) was combined with EDC (600 mg, 3.14 mmol), HOBt (600 mg, 3.92 mmol), and N-hydroxy-benzamidine (292 mg, 2.14 mmol). Dichloromethane (10 mL) was added and then 4-methylmorpholine (1 mL). The reaction mixture was stirred at ambient temperature for 16 h. After dilution with ethyl acetate (200 mL), the solution was washed with water (30 mL), saturated aqueous NaHCO₃ solution and brine, dried with MgSO₄ and evaporated under vacuum. The crude product was dissolved in pyridine (10 mL) and heated at 80° C. for 15 h. The pyridine was evaporated under vacuum and the residue was purified by flash chromatography on silica gel (eluent: ethyl acetate) to yield 2(S)-tert-butoxycarbonylamino-1-(3-phenyl-[1,2,4]oxadiazol-5-yl)-butan-1-ol (290 mg). (S)-tert-Butoxycarbonylamino-1 3-phenyl-[1,2,4]oxadiazol-5-yl)butan-1-ol (145 mg, 0.41 mmol) was dissolved in CH₂Cl₂ (4 mL) and TFA (4 mL) was added. After stirring for 1 h, the reaction mixture was evaporated to dryness to yield 2(S)-amino-1-(3-phenyl-[1,2,4]oxadiazol-5-yl)-butan-1-ol.

### Reference L

### Synthesis of 2(S)-amino-1-(2-phenyl-[1,3]dithian-2-yl)-hexan-1-ol

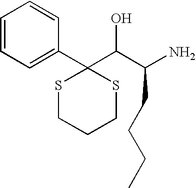 Step 1

2-Phenyl-1,3-dithiane (Aldrich) (3.79 g; 19.3 mmol) was mixed with dry distilled THF (20 mL) under a nitrogen atmosphere. The solution was cooled to −60° C. and n-buty lithium (1.6M in pentane, 1.56 mmol, 9.74 mL) was added slowly by syringe. The reaction mixture was warmed to −20° C. and held at that temperature for 30 min., and then held at −10° C. for 15 min. The yellow solution was cooled to −78° C. and (1(S)-formylpentyl)-carbamic acid tert-butyl ester (1.6 g, 1.4 mmol, in 5 mL THF) was added rapidly (over 20 seconds) and 60 seconds later a mixture of acetic acid (2 mL) and THF (5 mL) was added rapidly. After warming to 23° C., the solution was concentrated at reduced pressure. Excess 2-phenyl-1,3-dithiane was removed by its crystallization away from the desired product using a minimum of ethyl acetate in hexane. The mother liquors were concentrated and chromatographed using a hexane-ethyl acetate gradient to afford {1(S)-[hydroxy-(2-phenyl-[1,3]dithian-2-yl)-methyl]pentyl}carbamic acid tert-butyl ester. (1.7 g, 56% yield).

Step 2

To {1(S)-[hydroxy-(2-phenyl-[1,3]dithian-2-yl)methyl]pentyl}carbamic acid tert-butyl ester (608 mg, 1.47 mmol) in dioxane (2.7 mL) at 10° C. was added hydrochloric acid (2.7 mL, 4 M in dioxane). The solution was warmed to 23° C. After 3 h, the solution was diluted with toluene (5 ml) and concentrated under reduced pressure. The gummy solid was washed with diethyl ether resulting in the hydrochloride salt of 2(S)-amino-1-(2-phenyl-[1,3]dithian-2-yl)-hexan-1-ol (414 mg) as a free flowing solid after removal of excess ether under reduced pressure.

### Reference M

### Synthesis of 3-amino-4-hydroxopyrrolidine-1-carboxylic acid tert-butyl ester

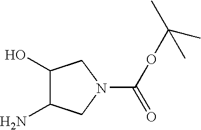

6-Oxa-3-aza-bicyclo[3.1.0]hexane-3-carboxylic acid tert-butyl ester (12.1 g, 65.3 mmol) was dissolved in a 8:1 methanol/water mixture (108 mL). Ammonium chloride (15 g) and sodium azide (21.4 g, 329 mmol) was added and the reaction mixture was heated at 60° C. overnight. After dilution with ether (500 mL), the reaction mixture was washed with saturated aqueous NaHCO₃ (200 mL) and brine (200 mL), dried with MgSO₄ and evaporated under vacuum. The crude product was dissolved in methanol (200 mL). 10% Palladium on activated carbon (1.5 g) was added and the reaction mixture was stirred at ambient temperature under a hydrogen atmosphere until TLC analysis showed the disappearance of the starting material. The reaction mixture was filtered through a pad of Celite™ and evaporated to dryness under vacuum. The product was purified by flash chromatography on silica gel using 5% methanol in ethyl acetate to 20% methanol, 3% triethylamine in ethyl acetate to give 4.3 g of 3-amino-4-hydroxy-pyrrolidine-1-carboxylic acid tert-butyl ester as yellowish solid.

### Reference N

### Synthesis of 2-amino-2-methyl-1-oxazolo[4,5-b]pyridin-2-yl-propan-1-ol

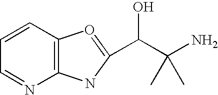 Step 1

2-Amino-2-methyl-1-propanol (17.8 g, 200 mmol) was dissolved in a mixture of water and dioxane (100 mL) and cooled to 0° C. NaOH (8 g, 200 mmol) and di-tert-butyldicarbonate (52.4 g, 240 mmol) were added and the reaction was allowed to warm to room temperature with stirring for 2 h. After removing the dioxane, the residue was extracted with EtOAc, washed with brine, dried with anhydrous MgSO₄, filtered and concentrated to yield 35 g of 2-Boc-amino-2-methyl-1-propanol.

Step 2

A solution of oxalyl chloride (15.24 g, 120 mmol) in 200 mL of CH₂Cl₂ was stirred and cooled to −60° C. followed by the drop wise addition of dimethylsulfoxide (19.7 g, 252 mmol) in of CH₂Cl₂(60 mL). After 10 min, a solution of 2-Boc-amino-2-methyl-1-propanol (18.9 g, 100 mmol) in CH₂Cl₂ (60 ml) was added drop wise at −70° C. The reaction mixture was allowed to warm to −40° C. for 10 min followed by cooling to −70° C. before the addition of a solution of triethylamine (28.28 g, 280 mmol) in CH₂Cl₂ (60 mL). The reaction mixture was allowed to warm to room temperature over a two-hour period and saturated sodium dihydrogen phosphate (40 mL) was added. The organic layer was washed with brine and dried over MgSO₄. The solvent was removed to yield 2-Boc-amino-2-methylpropionaldehyde (17.3 g).

Step 3

A mixture of 2-amino-3-hydroxypyridine (11 g, 100 mmol), triethylorthoformate (80 mL) and p-toluenesulfonic acid (61 mg) was heated at 140° C. for 8 h. Excess triethylorthoformate was removed under vacuum. The product was crystallized from ethyl acetate to yield 1-oxazolo[4,5-b]pyridine (9 g).

Step 4

To a stirred solution of the 1-oxazolo[4,5-b]pyridine (2.4 g, 20 mmol) in THF (100 mL) was added n-BuLi (1.6 M solution in 12.5 mL of hexane) dropwise under N₂ at −78° C. After 1 h, MgBr.Et₂O (5.16 g, 20 mmol) was added and the reaction mixture was allowed to warm to −45° C. for 1 h before being treated with 2-Boc-amino-2-methylpropionaldehyde (2.24 g, 12 mmol) in THF (20 mL). The reaction mixture was stirred for 1 h, quenched with saturated NH₄Cl, and extracted with ethyl acetate. The organic layer was washed with brine, dried with MgSO₄ and concentrated. The residue was purified by silica gel column chromatography to yield 2-Boc-amino-2-methyl-1-oxazolo[4,5-b]pyridin-2-yl-1-propanol (1.18 g).

Step 5

2-Boc-amino-2-methyl-1-oxazolo[4,5-b]pyridin-2-yl-1-propanol (156 mg, 0.508 mmol) and CH₂Cl₂ (5 mL) were mixed and TFA (0.5 mL) was added at room temperature. After stirring for 1 h, the solvent and excess TFA were removed under vacuum to produce 2-amino-2-methyl-1-oxazolo[4,5-b]pyridin-2-yl-propan-1-ol. TFA salt (165 mg).

### Reference O

### Synthesis of 2(S)-amino-1-(5-methoxymethyl-[1,3,4]oxadiazol-2-yl)butan-1-ol

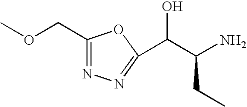 Step 1

(S)-(+)-2-amino-1-butanol (50 g, 561 mmol) in a mixture of water and dioxane (200 mL:200 mL) was cooled to 0° C. and mixed with NaOH (26.9 g, 673 mmol) and di-tert-butyl-dicarbonate (146.96 g, 673 mmol) was added. After the addition, the reaction was allowed to warm to room temperature and the reaction mixture was stirred for 2 h. After removing the dioxane, the residue was extracted with EtOAc, then washed with brine and dried with anhydrous MgSO₄, filtered and concentrated. Without further purification, the crude 2(S)-Boc-amino-1-butanol (120 g) was used for next step reaction.

Step 2

A solution of oxalyl chloride (40.39 g, 265 mmol) in CH₂Cl₂ (700 mL) was stirred and cooled to 60° C. Dimethylsulfoxide (51.7 g, 663 mmol) in CH₂Cl₂ (100 mL) was added dropwise. After 10 min, a solution of(S)-2-Boc-amino-1-butanol (50 g, 265 mmol) in CH₂Cl₂ (100 mL) was added dropwise at −70° C. The reaction mixture was allowed to warm to −40° C. for 10 min and then cooled to −70° C. again. A solution of triethylamine (74.9 g, 742 mmol) in CH₂Cl₂ (100 mL) was added. The reaction mixture was allowed to warm to room temperature over 2 h. Saturated sodium dihydrogen phosphate (100 mL) was added, and then the organic layer was washed with brine and dried over MgSO₄. The solvent was removed to yield 45 g of 2(S)-Boc-amino-butyraldehyde.

Step 3

A mixture of methyl methoxyacetate (52 g, 500 mmol), hydrazine hydrate (30 mL) was heated to reflux for 8 h. Excess hydrazine and water were removed under vacuum. The residue was extracted with n-butanol, dried with Na₂SO₄. Excess n-butanol was removed to yield 45 g of hydrazide.

Step 4

A mixture of above hydrazide (45 g), triethylorthoformate (146 mL) and p-toluenesulfonic acid (61 mg) was heated at 140° C. for 8 h. Excess triethylorthoformate was removed under vacuum. The product was purified by silica gel column chromatography to yield 4.6 g of 2-methoxymethyl-1,3,4-oxadiazole.

Step 5

To a stirred solution of 2-methoxymethyl-1,3,4-oxadiazole (4.6 g, 40 mmol) in THF (100 mL) was added n-BuLi (1.6 M solution in 25.2 mL of hexane) dropwise under N₂ at −78° C. After 1 h, MgBr.Et₂O (10.4 g, 40.3 mmol) was added and the reaction mixture was allowed to warm to −45° C. for 1 h before being treated with 2(S)-Boc-amino-butyraldehyde (5.28 g, 28.25 mmol) in THF (20 mL). The reaction mixture was stirred for 1 h, quenched with saturated NH₄Cl, and extracted with ethyl acetate. The organic layer was washed with brine, dried with MgSO₄ and concentrated. The residue was purified by silica gel column chromatography to yield 2(S)-Boc-amino-1-(5-methoxymethyl-1,3,4-oxadiazol-2-yl)-1-butanol (500 mg).

Step 6

2(S)-Boc-Amino-1-(5-methoxymethyl-1,3,4-oxadiazol-2-yl)-1-butanol (500 mg, 1.66 mmol), and CH₂Cl₂ (5 mL) were mixed and TFA (0.5 mL) was added at room temperature. After stirring for 1 h, the solvent and excess TFA were removed under vacuum to produce 2(S)-amino-1-(5-methoxymethyl-[1,3,4]oxadiazol-2-yl)-butan-1-ol. TFA salt (340 mg).

### Reference P

### Synthesis of 2(S)-amino-1-(5-phenyl-[1,3,4]oxadiazol-2-yl)-butan-1-ol

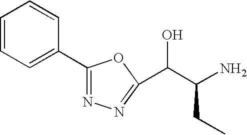 Step 1

A mixture of the benzoic hydrazide (22.5 g, 165 mmol), triethylorthoformate (150 mL) and p-toluenesulfonic acid (300 mg) was heated at 120° C. for 12 h. Excess triethylorthoformate was removed under vacuum and the residue was purified by silica gel column chromatography to produce 2-phenyl-1,3,4-oxadiazole (14.5 g).

Step 2

To a stirred solution of the 2-phenyl-[1,3,4]oxadiazole (10 g, 68.5 mmol) in THF (100 mL) was added n-BuLi (1.6 M solution in 42.8 mL of hexane) dropwise under N₂ at −78° C. After 1 h, MgBr.Et₂O (17.69 g, 68.5 mmol) was added and the reaction mixture was allowed to warm to −45° C. for 1 h before being treated with 2(S)-Boc-aminobutyraldehyde (7.8 g, 41 mmol) in THF (20 mL). The reaction mixture was stirred for 1 h, quenched with saturated NH₄Cl, and extracted with ethyl acetate. The organic layer was washed with brine, dried with MgSO₄ and concentrated. The residue was purified by silica gel column chromatography to yield 2-(2(S)-Boc-amino-1-hydroxybutyl)-5-phenyl-1,3,4-oxadiazole (9.7 g).

Step 3

2-(2(S)-Boc-amino-1-hydroxybutyl)-5-phenyl-1,3,4-oxadiazole (505 mg, 1.5 mmol) and CH₂Cl₂ (5 mL) were mixed and TFA (1 mL) was added at room temperature. After stirring for 1 h, the solvent and excess TFA were removed under vacuum to produce 530 mg of (S)-2-amino-1-(5-phenyl-[1,3,4]oxadiazol-2-yl)-1-butanol TFA salt.

### Reference Q

### Synthesis of 2(S)-amino-1-oxazolo[4,5-b]pyridin-2-yl-butan-1-ol

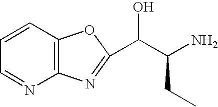 Step 1

A mixture of 2-amino-3-hydroxypyridine (25 g, 227 mmol), triethylorthoformate (75 mL) and p-toluenesulfonic acid (61 mg) was heated at 140° C. for 8 h. Excess triethylorthoformate was removed under vacuum. The product was crystallized from ethyl acetate to yield 22.5 g of oxazolo[4,5-b]pyridine.

Step 2

To a stirred solution of the oxazolo[4,5-b]pyridine (12 g, 100 mmol) in THF (300 mL) was added n-BuLi (1.6 M solution in 62.5 mL of hexane) drop wise under N₂ at −78° C. After 1 h, MgBr.Et₂O (25.8 g, 100 mmol) was added and the reaction mixture was allowed to warm to −45° C. for 1 h before being treated with (S)-2-Boc-aminobutylaldehyde (11.46 g, 60 mmol) in THF (50 mL). The reaction mixture was stirred for 1 h, quenched with saturated NH₄Cl, and extracted with ethyl acetate. The organic layer was washed with brine, dried with MgSO₄ and concentrated. The residue was purified by silica gel column chromatography to yield 2(S)-Boc-amino-1-(oxazolo[4,5-b]pyridin-2-yl)-1-butanol (14.1 g).

Step 3

2(S)-Boc-Aamino-1-(oxazolo[4,5-b]pyridin-2-yl)-1-butanol (311 mg, 1 mmol) and MeCl₂ (5 mL) were mixed and TFA (1 mL) was added at room temperature. After stirring for 1 h, the solvent and excess TFA were removed under vacuum to produce 355 mg of (S)-2-amino-1-oxazolo[4,5-b]pyridin-2-yl-butan-1-ol TFA salt.

### Reference R

### Synthesis of 2(S)-amino-1-(5-pyridin-4-yl-[1,3,4]oxadiazol-2-yl)butan-1-ol

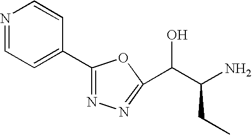 Step 1

A mixture of the isonicotinic hydrazide (13.7 g, 100 mmol), triethylorthoformate (60 mL) and p-toluenesulfonic acid (30 mg) was heated at 130° C. for 12 h. Excess triethylorthoformate was removed under vacuum. The crude was crystallized from ethyl acetate to give 14.8 g of 5-pyridin-4-yl-[1,3,4]oxadiazole.

Step 2

To a stirred solution of the 5-pyridin-4-yl-[1,3,4]oxadiazole (11.5 g, 78.2 mmol) in THF (300 mL) was added HMPA (5 ML) and n-BuLi (1.6 M solution in 48.9 mL of hexane) dropwise under N₂ at −78° C. After 1 h, MgBr.Et₂O (20.2 g, 78.2 mmol) was added and the reaction mixture was allowed to warm to 45° C. for 1 h before being treated with 2-Boc-amino-butyraldehyde (9.7 g, 50.8 mmol) in THF (5 mL). The reaction mixture was stirred for 1 h, quenched with saturated NH₄Cl, and extracted with ethyl acetate. The organic layer was washed with brine, dried with MgSO₄ and concentrated. The residue was purified with silica gel column chromatography to yield 2(S)-Boc-amino-1-(5-pyridin-4-yl-[1,3,4]oxadiazol-2-yl)-butan-1-ol (3.5 g).

Step 3

2(S)-Boc-Amino-1-(5-pyridin-4-yl-[1,3,4]oxadiazol-2-yl)-butan-1-ol (334 mg, 1 mmol) and MeCl₂ (5 mL) were mixed and TFA (0.5 mL) was added at room temperature. After stirring for 1 h, the solvent and excess TFA were removed under vacuum to produce 350 mg of 2(S)-amino-1-(5-pyridin-4-yl-[1,3,4]oxadiazol-2-yl)-butan-1-ol TFA salt.

### Reference S

### Synthesis of 2(S)-amino-1-(5-pyridin-3-yl-[1,3,4]oxadiazol-2-yl)-butan-1-ol

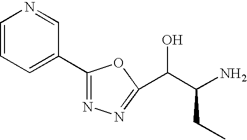 Step 1

To a stirred solution of the 3-[1,3,4]oxadiazol-2-yl-pyridine (5 g, 34 mmol) in THF (100 mL) was added HMPA (5 mL) and n-BuLi (1.6 M solution in hexane, 21.25 mL) drop wise under N₂ at −78° C. After 1 h, MgBr.Et₂O (8.77 g, 34 mmol) was added and the reaction mixture was allowed to warm to −45° C. for 1 h before being treated with 2(S)-Boc-aminobutyraldehyde (4.22 g, 22.1 mmol) in THF (20 mL). The reaction mixture was stirred for 1 h, quenched with saturated NH₄Cl, and extracted with ethyl acetate. The organic layer was washed with brine, dried with MgSO₄ and concentrated. The residue was purified with silica gel column chromatography to yield 2(S)-Boc-amino-1-(5-pyridin-3-yl-[1,3,4]oxadiazol-2-yl)-butan-1-ol (1.5 g).

Step 2

2(S)-Boc-Amino-1-(5-pyridin-3-yl-[1,3,4]oxadiazol-2-yl)-butan-1-ol (167 mg, 0.5 mmol) and CH₂Cl₂ (5 mL) were mixed and TFA (0.5 mL) was added at room temperature. After stirring for 1 h, the solvent and excess TFA were removed under vacuum to produce 180 mg of 2(S)-amino-1-(5-pyridin-3-yl-[1,3,4]oxadiazol-2-yl)-butan-1-ol TFA salt.

### Reference T

### Synthesis of 2(S)-amino-1-benzoxazol-2-ylbutan-1-ol hydrochloride

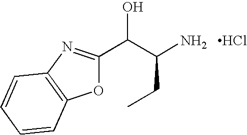 Step 1

To a solution of benzoxazole (28.6 g, 240 mmol) in toluene (150 mL) was added during ca 20 min., at about −4° C. a 2 M solution of isopropyl-magnesium chloride in THF (120 mL, 240 mmol). The red-brown mixture was stored at ca −4° C. and used as needed.

Step 2

To a solution of 2(S)-Boc-aminobutanol (50 g; 264 mmol) in dichloromethane (500 mL) and water (350 mL) were added at 20° C. TEMPO (0.01 eq), sodium bromide (1 eq) and sodium hydrogencarbonate (3 eq). The reaction mixture was stirred at 0° C. and diluted bleach (1.3 eq, 450 mL) was added over 40 min. The reaction mixture was stirred for 30 min. at 0° C. and then quenched with aq. thiosulfate. After decantation and extractions (dichloromethane), the organic phase was washed with brine, dried and concentrated in vacuo to dryness, giving 2(S)-(tert-butoxycarbonyl)-amino-butyraldehyde as a low-melting solid (38.1 g; yield: 77%).

Step 3

A solution of 2(S)-(tert-butoxycarbonyl)amino-butyraldehyde (30 g, 160 mmol) in toluene (150 mL) was added over 30 min. at −5° C. to a solution of Grignard reagent of benzoxazole (prepared as described in Step 1 above). The reaction mixture was stirred for 0.5 h at 0° C., then 2.5 h at RT. Quenching with 5% aq. acetic acid, washings with 5% aq. sodium carbonate, then brine and concentration to dryness gave crude 2(S)-(tert-butoxycarbonyl)-amino-1-benzoxazol-2-yl-propan-1-ol. The residue was diluted with toluene, and silica gel was added. The slurry was filtered. Elution by toluene removed the non-polar impurities. Then an 8/2 mixture of toluene and ethyl acetate desorbed the 2(S)-tert-butoxycarbonyl)-amino-1-benzoxazol-2-ylpropan-1-ol.

Step 4

To a solution of 2(S)-(tert-butoxycarbonyl)amino-1-benzoxazol-2-yl-propan-1-ol (26.3 g, 86 mmol) in isopropanol (118 mL) at 20-25° C. was added trimethylchlorosilane (1.4 eq). The solution was stirred for 5 h at 50° C. Concentration of the reaction mixture to 52 mL followed by addition of isopropyl ether (210 mL), filtration and drying under vacuum afforded 2(S)-amino-1-benzoxazol-2-yl-butan-1-ol hydrochloride salt as a grey solid (16.4 g; yield=79%; mixture of diastereomers.

### Reference U

### Synthesis of 2(S)-benzyloxycarbonylamino-3-(1-methylcyclopentyl)-propionic acid

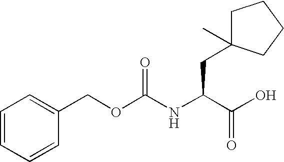 Step 1

1-Methylcyclopentanol (20 g, 0.2 mol) was added to hydrobromic acid (40 mL) at room temperature. After stirring for 1 h, the solution was extracted with hexane and the hexane was washed with brine and dried with magnesium sulfate. After concentration of the organic layer, 20.5 g of 1-methylcyclopentyl bromide was obtained.

Step 2

Tributyltin hydride (37.8 g, 130 mmol) was added at reflux to a 500 mL of flask charged with benzene (200 mL) was added Z-dehydro-Ala-OH (15 g, 64 mmol), 1-methylcyclopentanyl-bromide (20.5 g) and AIBN (1.9 g). After 2 h, the solvent was removed and the residue was purified by column chromatograph to yield 7.9 g of 2-benzyloxycarbonylamino-3-(1-methylcyclopentyl)-propionic acid methyl ester.

Step 3

2-Benzyloxycarbonylamino-3-(1-methylcyclopentyl)propionic acid methyl ester (7.6 g, 23.8 mmol) was dissolved in a mixture of acetonitrile (82 mL) and 0.2 M aqueous NaHCO₃ (158 mL) and Alcalase 2.4 L (1.1 mL) was added and the reaction mixture wa stirred vigorously for 8 h. The reaction mixture was then evaporated at 30° C. to remove acetonitrile, and the aqueous residue was washed with ether. The ethereal layer was concentrated to yield 2(R)-benzyloxycarbonyl-amino-3-1-methylcyclopentyl)propionic acid methyl ester (1.9 g). The aqueous phase was filtered with Celite, the pH was adjusted to 3 with 6 N HCl, and the solution was extracted with ethylacetate. The ethyl acetate layer was dried and evaporated to yield 2(S)-benzyloxycarbonylamino-3-(1-methylcyclopentyl)propionic acid (1.4 g).

### Reference V

### Synthesis of trifluoromethanesulfonic acid 2,2,2-trifluoro-1-(4-fluorophenyl)ethyl ester

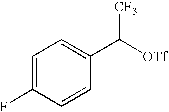 Step 1

To a stirred solution of 2,2,2,4′-tetrafluoroacetophone (10 g, 52.1 mmol) in methanol (50 mL) was added NaBH₄ (0.98 g, 26.5 mmol) at 0° C. After stirring at 25° C. for 2 h, the reaction mixture was quenched by adding 1N HCl (100 mL) and then extracted with ethyl ether. The ether extract was washed with brine, dried with MgSO₄, and concentrated to give 2,2,2-trifluoro-1-(4-fluorophenyl)ethanol (1 1.32 g) which was used in next step without further purification.

Step 2

NaH (640 mg, 16 mmol, 60% in mineral oil) was washed twice with hexane (20 mL) and then suspended in dried diethyl ether (20 mL). A solution of 2,2,2-trifluoro-1-(4-fluorophenyl)-ethanol (1.94 g, 10 mmol) in diethyl ether (10 mL) was added at 0° C. After stirring for 2 h at room temperature, a solution of trifluoromethanesulfonyl chloride (1.68 g, 10 mmol) in diethyl ether (10 mL) was added. After 2 h, the reaction mixture was quenched by adding a solution of NaHCO₃ and the product was extracted with diethyl ether. The extracts were washed with brine and dried, and the solvent was removed to yield trifluoro-methanesulfonic acid 2,2,2-trifluoro-1-(4-fluorophenyl)-ethyl ester (3.3 g).

Proceeding as described in Example V above, trifluoromethanesulfonic acid 2,2,2-trifluoro-1-phenylethyl ester was prepared.

### Reference W

### Synthesis of 2,2,2-trifluoro-1R-(4-fluorophenyl)ethanol

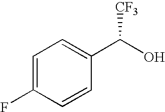

To a −78° C. toluene (25 mL)/dichloromethane (25 mL) solution of 2,2,2,4′-tetrafluoroacetophenone (2.5 g, 13.01 mmol) and 1M S-CBS catalyst (1.3 mL, 1.3 mmol) was added freshly distilled catecholborane (1.66 mL, 15.62 mmol). The reaction mixture was maintained at −78° C. for 16 h at which time 4N HCl (5 mL in dioxane) was added and the reaction mixture was allowed to warm to room temperature. The reaction mixture was diluted with ethyl acetate and washed with a saturated brine solution). The organic layer was dried over magnesium sulfate, filtered and concentrated to provide a solid. The solid was suspended in hexanes and filtered off. The hexanes filtrate containing the desired product was concentrated and the residue subjected to flash chromatography (10 hexanes: 1 ethylacetate) to provide the title compound as colorless oil (2.2 g, 87% yield). The ratio of enantiomers was determined to be 95:5 by chiral HPLC (Chiralcel OD column, 95 hexanes: 5 isopropanol mobile phase. Ret. time major product 6.757 min. Ret. time minor isomer 8.274 min.).

### Example 1

### Synthesis of N-[1((S)-(benzoxazol-2-ylcarbonyl)propyl]-3-(2-chlorophenyl)-2(S)-(2,2,2-trifluoro-1 (RS)-phenylethylamino)propionamide

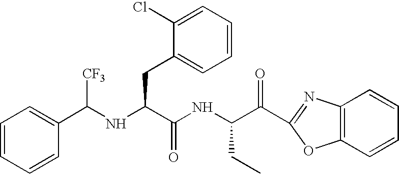 Step 1

2(S)-Amino-3-(2-chlorophenyl)propionic acid (1 g, commercially available) was dissolved in methanol (10 mL) and HCl gas was bubbled through the solution for 5 min. The reaction mixture was stirred at room temperature for 3 h and the solvent was evaporated using the rotavap to get 2(S)-amino-3-(2-chlorophenyl)propionic acid methyl ester hydrochloride (1.2 g).

Step 2

2,2,2-Trifluorol-phenylethanone (305 mg, 1.75 mmol) and 2(S)-amino-3-(2-chloro-phenyl)propionic acid methyl ester hydrochloride (500 mg, 1.75 mmol) were dissolved in DCM (10 mL). N,N-Diisopropylethylamine (1.2 mL, 7 mmol) was added followed by the addition 1M solution of TiCl₄ in DCM (1.75 mL, 1.75 mmol) and the reaction mixture was stirred for 18 h at room temperature. TiCl₄ (0.9 mL, 0.9 mmol) was added again and the solution was stirred at room temperature for 3 h. NaCNBH₃ (330 mg, 5.25 mmol) in MeOH (5 mL) was added and after stirring for 2 h, 1N NaOH solution (5 mL) was added. After 30 min, the suspension was filtered through celeite and the filtrate was extracted with ethylacetate. The organic layer was washed with brine and dried over MgSO₄. The solvent was evaporated to get methyl 3-(2-chlorophenyl)-2(S)-(2,2,2-trifluoro-1-phenylethylamino)propionate (600 mg) as a yellow solid which was used as such for the next step.

Step 3

Methyl 3-(2-chlorophenyl)-2(S)-(2,2,2-trifluoro-1-phenylethylamino)-propionate was dissolved in a mixture of MeOH (2 mL) and THF (5 mL) and 1N NaOH (4 mL) was added and the reaction mixture was stirred at room temperature for 4 h. The solvent was evaporated using a rotavap and the pH was adjusted to 6 using 1N HCl. The precipitated yellow solid was filtered and dried to give 3-(2-chlorophenyl)-2(S)-(2,2,2-trifluoro-1-phenylethylamino)propionic acid (500 mg).

Step 4

3-(2-Chlorophenyl)-2(S)-(2,2,2-trifluoro-1-phenylethylamino)propionic acid (150 mg, 0.42 mmol) was dissolved in DMF (1 mL). HATU (192 mg, 0.5 mmol), 2(S)-amino-1-benzoxazol-2-ylbutan-1-ol (86 mg, 0.42 mmol) and N,N-isopropylethylamine (146.3 μl, 0.84 mmol) were added and the reaction mixture was stirred at room temperature for 4 h. The reaction mixture was diluted with ethylacetate and was washed with water, 1N HCl, saturated solution of NaHCO₃ and brine. The organic layer was dried over MgSO₄ and was evaporated using rotavap to give N-[1(S)-(benzoxazol-2-ylhydroxymethyl)propyl]-3-(2-chlorophenyl)-2(S)-(2,2,2-trifluoro-1-phenyl-ethylamino)propionamide (350 mg) as a thick liquid.

Step 5

N-[1(R)-(Benzoxazol-2-ylhydroxymethyl)propyl]-3-(2-chlorophenyl)-2(S)-(2,2,2-trifluoro-1-phenylethylamino)propionamid was dissolved in methylene chloride (5 mL) and cooled to 0° C. and added NaBr (48 mg, 0.462 mmol), NaHCO₃ (40 mg, 0.462 mmol), TEMPO® (0.78 mg, 0.005 mmol) and bleach (1.5 mL, 0.84 mmol) in water (2 mL) were added and stirred for 1 h. The reaction mixture was diluted with ethylacetate and was washed with water, followed by brine and was dried over MgSO₄. The solvent was evaporated and was purified by preparative TLC by eluting with 50:50ethylacetate:hexanes to give the title compound (40 mg). LCMS: 542.2 (M−1)⁻¹, 544.0 (M+1)⁺¹.

Proceeding as described in Example 1 above, but substituting 2,2,2-trifluoro1-phenylethanone with 2,2,2-trifluoro-1-(4-fluorophenyl)ethanone gave N-[1((S)-(benzoxazol-2-ylcarbonyl)propyl]-3-(2-chlorophenyl)-2(S)-(2,2,2-trifluoro-1 (RS)-4-fluorophenylethylamino)-propionamide. LCMS: 560.2 (M−1)⁻¹, 562.1 (M+1)⁺¹, 583.9 (M+Na)⁺.

### BIOLOGICAL EXAMPLES

### Example 1

### Cathepsin B Assay

Solutions of test compounds in varying concentrations were prepared in 10 μL of dimethyl sulfoxide (DMSO) and then diluted into assay buffer (40 μL, comprising: N,N-bis(2-hydroxyethyl)-2-aminoethanesulfonic acid (BES), 50 mM (pH 6); polyoxyethylenesorbitan monolaurate, 0.05%; and dithiothreitol (DTT), 2.5 mM). Human cathepsin B (0.025 pMoles in 25 μL of assay buffer) was added to the dilutions. The assay solutions were mixed for 5-10 seconds on a shaker plate, covered and incubated for 30 min at room temperature. Z-FR-AMC (20 nMoles in 25 4 of assay buffer) was added to the assay solutions and hydrolysis was followed spectrophotometrically at (, 460 nm) for 5 min. Apparent inhibition constants (Kᵢ) were calculated from the enzyme progress curves using standard mathematical models.

Compounds of the invention were tested by the above-described assay and observed to exhibit cathepsin B inhibitory activity.

### Example 2

### Cathepsin K Assay

Solutions of test compounds in varying concentrations were prepared in 10 μL of dimethyl sulfoxide (DMSO) and then diluted into assay buffer (40 μL, comprising: MES, 50 mM (pH 5.5); EDTA, 2.5 mM; and DTT, 2.5 mM). Human cathepsin K (0.0906 pMoles in 25 μL of assay buffer) was added to the dilutions. The assay solutions were mixed for 5-10 seconds on a shaker plate, covered and incubated for 30 min at room temperature. Z-Phe-Arg-AMC (4 nMoles in 25 μL of assay buffer) was added to the assay solutions and hydrolysis was followed spectrophotometrically at (λ 460 nm) for 5 min. Apparent inhibition constants (Kᵢ) were calculated from the enzyme progress curves using standard mathematical models.

Compounds of the invention were tested by the above-described assay and observed to exhibit cathepsin K inhibitory activity.

### Example 3

### Cathepsin L Assay

Solutions of test compounds in varying concentrations were prepared in 10 μL of dimethyl sulfoxide (DMSO) and then diluted into assay buffer (40 μL, comprising: MES, 50 mM (pH 5.5); EDTA, 2.5 mM; and DTT, 2.5 mM). Human cathepsin L (0.05 pMoles in 25 μL of assay buffer) was added to the dilutions. The assay solutions were mixed for 5-10 seconds on a shaker plate, covered and incubated for 30 min at room temperature. Z-Phe-Arg-AMC (1 nMoles in 25 μL of assay buffer) was added to the assay solutions and hydrolysis was followed spectrophotometrically at (λ 460 nm) for 5 min. Apparent inhibition constants (Kᵢ) were calculated from the enzyme progress curves using standard mathematical models.

Compounds of the invention were tested by the above-described assay and observed to exhibit cathepsin L inhibitory activity.

### Example 4

### Cathepsin S Assay

Solutions of test compounds in varying concentrations were prepared in 10 μL of dimethyl sulfoxide (DMSO) and then diluted into assay buffer (40 μL, comprising: MES, 50 mM (pH 6.5); EDTA, 2.5 mM; and NaCl, 100 mM); β-mercaptoethanol, 2.5 mM; and BSA, 0.00%. Human cathepsin S (0.05 pMoles in 25 μL of assay buffer) was added to the dilutions. The assay solutions were mixed for 5-10 seconds on a shaker plate, covered and incubated for 30 min at room temperature. Z-Val-Val-Arg-AMC (4 nMoles in 25 μL of assay buffer containing 10% DMSO) was added to the assay solutions and hydrolysis was followed spectrophotometrically (at λ 460 nm) for 5 min. Apparent inhibition constants (Kᵢ) were calculated from the enzyme progress curves using standard mathematical models.

Compounds of the invention were tested by the above-described assay and observed to exhibit cathepsin S inhibitory activity.

### Example 5

### Cathepsin F Assay

Solutions of test compounds in varying concentrations were prepared in 10 μL of dimethyl sulfoxide (DMSO) and then diluted into assay buffer (40 μL, comprising: MES, 50 mM (pH 6.5); EDTA, 2.5 mM; and NaCl, 100 mM); DTT, 2.5 mM; and BSA, 0.01%. Human cathepsin F (0.1 pMoles in 25 μL of assay buffer) was added to the dilutions. The assay solutions were mixed for 5-10 seconds on a shaker plate, covered and incubated for 30 min at room temperature. Z-Phe-Arg-AMC (2 nMoles in 25 μL of assay buffer containing 10% DMSO) was added to the assay solutions and hydrolysis was followed spectrophotometrically (at λ 460 nm) for 5 min. Apparent inhibition constants (Kᵢ) were calculated from the enzyme progress curves using standard mathematical models.

Compounds of the invention were tested by the above-described assay and observed to exhibit cathepsin F inhibitory activity.

### Example 1

### Representative Pharmaceutical Formulations Containing a Compound of Formula (I)

ORAL FORMULATION

| | | |
|---|---|---|
| Compound of Formula (I) | 10-100 | mg |
| Citric Acid Monohydrate | 105 | mg |
| Sodium Hydroxide | 18 | mg |
| Flavoring | | |

| | |
|---|---|
| Water | q.s. to 100 mL |

INTRAVENOUS FORMULATION

| Compound of Formula (I) | 0.1-10 | mg |
|---|---|---|
| Dextrose Monohydrate | q.s. to make isotonic | |
| Citric Acid Monohydrate | 1.05 | mg |
| Sodium Hydroxide | 0.18 | mg |
| Water for Injection | q.s. to 1.0 | mL |

TABLET FORMULATION

| | |
|---|---|
| Compound of Formula (I) | 1% |
| Microcrystalline Cellulose | 73% |
| Stearic Acid | 25% |
| Colloidal Silica | 1% |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

We claim:

1. A compound of Formula (I): 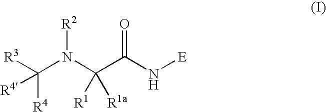
wherein:
E is:
(i) —C(R⁵)(R⁶)X¹ where X¹ is —CHO, —C(R⁷)(R⁸)CF₃, —C(R⁷)(R⁸)CF₂CF₂R⁹, —C(R⁷)(R⁸)R¹⁰, —CH═CHS(O)₂R¹⁰, —C(R⁷)(R⁸)C(R⁷)(R⁸)OR¹⁰, —C(R⁷)(R⁸)CH₂OR¹⁰, —C(R⁷)(R⁸)C(R⁷)(R⁸)R¹⁰, —C(R⁷)(R⁸)CH₂N(R¹¹)SO₂R¹⁰, —C(R⁷)(R⁸)CF₂C(O)NR¹⁰R¹¹, —C(R⁷)(R⁸)C(O)NR¹⁰R¹¹, —C(R⁷)(R⁸)C(O)N(R¹¹)(CH₂)₂OR¹¹, or —C(R⁷)(R⁸)C(O)N(R¹¹)(CH₂)₂NR¹⁰R¹¹ where:
R⁵ is hydrogen or alkyl; and
R⁶ is selected from the group consisting of hydrogen, alkyl, haloalkyl, carboxyalkyl, alkoxycarbonylalkyl, cycloalkyl, cycloalkylalkyl, aryl, aralkyl, heteroaryl, heteroaralkyl, heterocyclyl, heterocyclylalkyl, cyano, -alkylene-X²—R¹² (where X² is —O—, —NR¹³—, —CONR¹³—, —S(O)ₙ₁—, —NHCO—, —CO—, or —C(O)O— where n1 is 0-2, and R¹² and R¹³ are independently hydrogen, alkyl, haloalkyl, cycloalkyl, cycloalkylalkyl, aryl, aralkyl, heteroaryl, or heteroaralkyl) wherein the aromatic or alicyclic ring in R⁶ is optionally substituted with one, two, or three R^{a} independently selected from alkyl, haloalkyl, alkoxy, hydroxy, haloalkoxy, halo, carboxy, alkoxycarbonyl, amino, monosubstituted amino, disubstituted amino, nitro, aryloxy, benzyloxy, acyl, or arylsulfonyl and further where the aromatic or alicyclic ring in R^{a} is optionally substituted with one or two substituents independently selected from alkyl, halo, alkoxy, haloalkyl, haloalkoxy, hydroxy, amino, alkylamino, dialkylamino, carboxy, or alkoxycarbonyl; or
R⁵ and R⁶ taken together with the carbon atom to which both R⁵ and R⁶ are attached form (i) cycloalkylene optionally substituted with one or two R^{b} independently selected from alkyl, halo, alkylamino, dialkylamino, aryl, aralkyl, cycloalkyl, cycloalkylalkyl, heteroaryl, heteroaralkyl, alkoxycarbonyl, or aryloxycarbonyl, or (ii) heterocyclylalkylene optionally substituted with one to four R^{c} which are independently selected from alkyl, haloalkyl, hydroxy, hydroxyalkyl, alkoxyalkyl, alkoxyalkyloxyalkyl, aryloxyalkyl, heteroaryloxyalkyl, aminoalkyl, acyl, aryl, aralkyl, heteroaryl, heteroaralkyl, heterocyclyl, heterocyclylalkyl, cycloalkyl, cycloalkylalkyl, —S(O)ₙ₂R¹⁴, -alkylene-S(O)ₙ₂—R¹⁵, —COOR¹⁶, -alkylene-COOR¹⁷, —CONHR¹⁸R¹⁹, or -alkylene-CONHR²⁰R²¹ (where n2 is 0-2 and R¹⁴-R¹⁷, R¹⁸ and R²⁰ are independently hydrogen, alkyl, haloalkyl, aryl, aralkyl, heteroaryl, heteroaralkyl, cycloalkyl, cycloalkylalkyl, or heterocyclyl and R¹⁹ and R²¹ are independently hydrogen or alkyl) and further wherein the aromatic or alicyclic ring in the groups attached to cycloalkylene or heterocyclylalkylene is optionally substituted with one, two, or three substituents independently selected from alkyl, haloalkyl, alkoxy, hydroxy, haloalkoxy, halo, carboxy, alkoxycarbonyl, amino, monosubstituted amino, disubstituted amino, or acyl;
R⁷ is hydrogen or alkyl;
R⁸ is hydroxy; or
R⁷ and R⁸ together form oxo;
R⁹ is hydrogen, halo, alkyl, aralkyl or heteroaralkyl;
R¹⁰ is hydrogen, alkyl, aryl, aralkyl, heteroaryl, heteroaralkyl, cycloalkyl, cycloalkylalkyl, heterocyclyl, or heterocyclylalkyl wherein the aromatic or alicyclic ring in R¹⁰ is optionally substituted with one, two, or three R^{d} independently selected from alkyl, haloalkyl, alkoxy, cycloalkyl, hydroxy, haloalkoxy, halo, carboxy, alkoxycarbonyl, aryl, heteroaryl, amino, monosubstituted amino, disubstituted amino, or acyl and further wherein the aromatic or alicyclic ring in R^{d} is optionally substituted with one, two, or three substitutents independently selected from alkyl, haloalkyl, alkoxy, haloalkoxy, halo, hydroxy, carboxy, alkoxycarbonyl, amino, alkylamino, or dialkylamino; and
R¹¹ is hydrogen or alkyl; or
(ii) a group of formula (a): 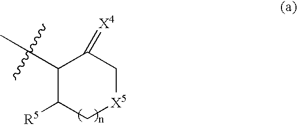
where:
n is 0, 1, or 2;
X⁴ is selected from —NR²², —S—, or —O— where R²² is hydrogen, alkyl, or alkoxy; and
X⁵ is —O—, —S—, —SO₂—, or —NR²³ where R²³ is selected from hydrogen, alkyl, haloalkyl, hydroxyalkyl, alkoxyalkyl, aryloxyalkyl, heteroaryloxyalkyl, aminoalkyl, acyl, aryl, aralkyl, heteroaryl, heteroaralkyl, cycloalkyl, cycloalkylalkyl, —S(O)₂R²⁴, -alkylene-S(O), —R²⁵, —COR²⁶, alkylene-COR²⁷, —CONR²⁸R²⁹, or -alkylene-CONR³⁰R³¹ (where n3 is 0-2, R²⁴-R²⁷, R²⁸ and R³⁰ are independently hydrogen, alkyl, haloalkyl, aryl, aralkyl, heteroaryl, heteroaralkyl, cycloalkyl, cycloalkylalkyl, heterocyclyl, or heterocyclylalkyl, and R²⁹ and R³¹ are independently hydrogen or alkyl) where the aromatic or alicyclic ring in the groups attached to X⁵ is optionally substituted with one, two, or three substituents independently selected from alkyl, haloalkyl, alkoxy, haloalkoxy, halo, hydroxy, amino, alkylamino, dialkylamino, carboxy, or alkoxycarbonyl; and
R⁵ is as defined above;
R¹ is hydrogen or alkyl;
R^{1a} is hydrogen, alkyl, haloalkyl, cycloalkyl, cycloalkylalkyl, aryl, aralkyl, heteroaryl, heteroaralkyl, heterocyclylalkyl, or -alkylene-X⁶—R³² (wherein X⁶ is —NR³³—, —O—, —S(O)ₙ₄—, —CO—, —COO—, —OCO—, —NR³³CO—, —CONR³³—, —NR³³SO₂—, —SO₂NR³³—, —NR³³COO—, —OCONR³³—, —NR³³CONR³⁴, or —NR³³SO₂NR³⁴— where R³³ and R³⁴ are independently hydrogen, alkyl, or acyl, n4 is 0-2, and R³² is hydrogen, alkyl, haloalkyl, cycloalkyl, cycloalkylalkyl, aryl, aralkyl, heteroaryl, heteroaralkyl, heterocyclyl, or heterocyclylalkyl) wherein said alkylene chain in -alkylene-X⁶—R³² is optionally substituted with one to six halo and wherein the aromatic or alicyclic ring in R^{1a} is optionally substituted with one, two, or three R^{e} independently selected from alkyl, haloalkyl, alkoxy, hydroxy, haloalkoxy, halo, nitro, cyano, carboxy, alkoxycarbonyl, aryl, heteroaryl, cycloalkyl, cycloalkylalkyl, aralkyl, heteroaralkyl, heterocyclyl, amino, monosubstituted amino, disubstituted amino, acyl, or -(alkylene)ₘ—X⁷—R³⁵ (wherein X⁷ is NR³⁶—, —O—, —S(O)ₙ₅—, —CO—, —COO—, —OCO—, —NR³⁶CO—, —CONR³⁶—, —NR³⁶SO₂—, —SO₂NR³⁶—, —NR³⁶COO—, —OCONR³⁶—, —NR³⁶CONR³⁷—, or —NR³⁶SO₂NR³⁷— where R³⁶ and R³⁷ are independently hydrogen, alkyl, or acyl and m is 0 or 1, and n5 is 0-2, and R³⁵ is cycloalkyl, cycloalkylalkyl, aryl, aralkyl, heteroaryl, heteroaralkyl, heterocylyl or heterocyclylalkyl) wherein the aromatic or alicyclic ring in R^{e} is optionally substituted with one, two, or three substituents independently selected from alkyl, alkoxy, alkoxyalkyl, alkylsulfonyl, alkylsulfonylalkyl, alkylaminosulfonyl, acyl, halo, haloalkyl, haloalkoxy, cyano, nitro, hydroxy, hydroxyalkyl, carboxy, alkoxycarbonyl, aryl optionally substituted with alkoxy or halo, aralkyl optionally substituted with alkoxy or halo, aryloxy optionally substituted with alkoxy or halo, heteroaryl optionally substituted with alkoxy or halo, or heteroaralkyl optionally substituted with alkoxy or halo, amino, aminosulfonyl, alkylamino, dialkylamino, or alkynyl optionally substituted with hydroxy, aryl, or heteroaryl; or
R¹ and R^{1a} together with the carbon atoms to which they are attached form cycloalkylene or heterocyclylalkylene ring wherein said cycloalkylene or heterocyclylalkylene is optionally substituted with one or two R^{f} independently selected from alkyl, halo, hydroxyalkyl, keto, or —SO₂R where R is alkyl, cycloalkyl, cycloalkylalkyl, aryl, aralkyl, heteroaryl or heteroaralkyl and further where the aromatic or alicylic ring in R^{f} is optionally substituted with one, two, or three substitutents independently selected from alkyl, alkoxy, haloalkyl, haloalkoxy, hydroxy, halo, carboxy, or alkoxycarbonyl;
R² is hydrogen or alkyl;
R³ is hydrogen, alkyl, haloalkyl, cycloalkyl, aryl, aralkyl, heteroaryl, heteroaralkyl, heterocyclyl, heterocyclylalkyl, or -alkylene-X⁸—R³⁸ (wherein X⁸ is —NR³⁹—, —O—, —S(O)ₙ₆—, —CO—, —COO—, —OCO—, —NR³⁹CO—, —CONR³⁹—, —NR³⁹SO₂—, —SO₂NR³⁹—, —NR³⁹COO—, —OCONR³⁹—, —NR³⁹CONR⁴⁰—, or —NR³⁹SO₂NR⁴⁰— where R³⁹ and R⁴⁰ are independently hydrogen, alkyl, or acyl, n6 is 0-2, and R³⁸ is hydrogen, alkyl, haloalkyl, cycloalkyl, cycloalkylalkyl, aryl, aralkyl, heteroaryl, or heteroaralkyl) wherein the aromatic or alicyclic rings in R³ are optionally substituted with one, two, or three R^{g} independently selected from alkyl, halo, hydroxy, alkoxy, haloalkyl, haloalkoxy, oxo, cyano, nitro, acyl, acyloxy, aryl, aralkyl, heteroaryl, heteroaralkyl, cycloalkyl, cycloalkylalkyl, heterocyclyl, heterocyclylalkyl, aryloxy, benzyloxy, carboxy, alkoxycarbonyl, aryloxycarbonyl, carbamoyl, alkylthio, alkylsulfinyl, alkylsulfonyl, arylthio, arylsulfonyl, arylsulfinyl, alkoxycarbonylamino, aryloxycarbonylamino, alkylcarbamoyloxy, arylcarbamoyloxy, alkylsulfonylamino, arylsulfonylamino, aminosulfonyl, alkylaminosulfonyl, dialkylaminosulfonyl, arylaminosulfonyl, aralkylaminosulfonyl, aminocarbonyl, arylaminocarbonyl, aralkylaminocarbonyl, amino, monosubstituted or disubstituted amino, and further wherein the aromatic or alicyclic ring in R^{g} is optionally substituted with one, two, or three R^{h} wherein R^{h} is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, amino, monosubstituted amino, dialkylamino, aryl, heteroaryl, cycloalkyl, carboxy, carboxamido, or alkoxycarbonyl; and
R⁴ is haloalkyl;
R^{4′} is hydrogen, alkyl, alkoxyalkyl, or haloalkyl; or
R³ and R^{4′} together with the carbon atom to which they are attached form cycloalkylene or heterocyclylalkylene wherein said cycloalkylene is optionally substituted with one or two substituents independently selected from alkyl, haloalkyl, hydroxy, or alkoxy and heterocyclylalkylene is optionally substituted with one to three substituents independently selected from alkyl, haloalkyl, hydroxy, alkoxy, carboxy, alkoxycarbonyl, alkylsulfonyl, aryl, heteroaryl, or hydroxyalkyl; or
a pharmaceutically acceptable salts thereof;
provided that when E is a group of formula (a), then: (i) R^{1a} is not hydrogen, alkyl, haloalkyl, cycloalkyl, or cycloalkylalkyl and (ii) R¹ and R^{1a} together with the carbon atoms to which they are attached do not form cycloalkylene or heterocyclylalkylene ring wherein said cycloalkylene or heterocyclylalkylene is optionally substituted with one or two R¹ independently selected from alkyl, halo, hydroxyalkyl, keto, or —SO₂R where R is alkyl, cycloalkyl, cycloalkylalkyl, aryl, aralkyl, heteroaryl or heteroaralkyl and further where the aromatic or alicylic ring in R^{f} is optionally substituted with one, two, or three substitutents independently selected from alkyl, alkoxy, haloalkyl, haloalkoxy, hydroxy, halo, carboxy, or alkoxycarbonyl.

2. The compound of claim 1 wherein E is —C(R⁵)(R⁶)X¹ in which:
R⁵ is hydrogen or alkyl; and
R⁶ is hydrogen, alkyl, -(alkylene)-OR¹² (where R¹² is hydrogen, alkyl or haloalkyl), cycloalkyl, cycloalkylalkyl, aryl, aralkyl, heteroaryl, heteroaralkyl, heterocyclyl, heterocyclylalkyl wherein the aromatic or alicyclic ring in aryl, aralkyl, heteroaryl, heteroaralkyl, heterocyclyl or heterocyclylalkyl is optionally substituted with one, two, or three R^{a} independently selected from alkyl, haloalkyl, alkoxy, hydroxy, haloalkoxy, halo, carboxy, alkoxycarbonyl, amino, monosubstituted amino, disubstituted amino, or acyl.

3. The compound of claim 1 wherein:
R⁵ is hydrogen;
R⁶ is alkyl; and
X¹ is —CHO, —C(O)R¹⁰, —C(O)CF₃, —C(O)CF₂CF₂R⁹—CH═CHS(O)₂R¹⁰, —C(O)CF₂C(O)NR¹⁰R¹¹, —C(O)C(O)NR¹⁰R¹¹, —C(O)CH₂OR¹⁰, —C(O)CH₂N(R¹¹)SO₂R¹⁰, —C(O)C(O)N(R¹¹)(CH₂)₂OR¹¹, —C(O)C(O)N(R¹¹)(CH₂)₂NHR¹¹ or —C(O)C(O)R¹⁰; wherein R¹⁰ is alkyl, aryl, aralkyl, heteroaryl, heteroaralkyl, cycloalkylalkyl or heterocyclylalkyl wherein the aromatic ring is optionally substituted with R^{d} selected from heteroaryl, aryl, or alkyl, R¹¹ is hydrogen or alkyl and R⁹ is halo.

4. The compound of claim 1 wherein:
Preferably, E is —CHR⁶C(O)R¹⁰ where R⁶ is alkyl and R¹⁰ is heteroaryl optionally substituted with one or two R^{d} independently selected from alkyl, haloalkyl, alkoxy, cycloalkyl, hydroxy, haloalkoxy, halo, carboxy, alkoxycarbonyl, aryl, heteroaryl, amino, monosubstituted amino, disubstituted amino, or acyl wherein the aromatic or alicyclic ring in R^{d} is optionally substituted with one, two, or three substitutents independently selected from alkyl, haloalkyl, alkoxy, haloalkoxy, halo, hydroxy, carboxy, alkoxycarbonyl, amino, alkylamino, or dialkylamino.

5. The compound of claim 1 wherein:
E is —CHR⁶C(O)R¹⁰ where R⁶ is ethyl or propyl; and R¹⁰ is benzoxazol-2-yl, 4-azabenzoxazol-2-yl, 2-pyridin-3-yl-[1,3,4]-oxadiazol-5-yl, 2-pyridin-4-yl-[1,3,4]-oxadiazol-5-yl, 2-ethyl-[1,3,4]-oxadiazol-5-yl, 2-isopropyl-[1,3,4]-oxadiazol-5-yl, 2-tert-butyl-[1,3,4]-oxadiazol-5-yl, 2-phenyl-[1,3,4]-oxadiazol-5-yl, 2-methoxymethyl-[1,3,4]-oxadiazol-5-yl, 2-furan-2-yl-[1,3,4]-oxadiazol-5-yl, 2-thien-2-yl-[1,3,4]-oxadiazol-5-yl, 2-(4-methoxyphenyl)-[1,3,4]-oxadiazol-5-yl, 2-(2-methoxyphenyl)-[1,3,4]-oxadiazol-5-yl, 2-(3-methoxyphenyl)-[1,3,4]-oxadiazol-5-yl, 2-(2-trifluoromethoxyphenyl)-[1,3,4]-oxadiazol-5-yl, 2-(3-trifluoromethoxyphenyl)-[1,3,4]-oxadiazol-5-yl, 2-(4-trifluoromethoxyphenyl)-[1,3,4]-oxadiazol-5-yl, 2-(4-dimethylaminophenyl)-[1,3,4]-oxadiazol-5-yl, pyradizin-3-yl, pyrimidin-2-yl, 3-phenyl-[1,2,4]-oxadiazol-5-yl, 3-ethyl-[1,2,4]-oxadiazol-5-yl, 3-cyclopropyl-[1,2,4]-oxadiazol-5-yl, 3-thien-3-yl-[1,2,4]-oxadiazol-5-yl, 3-pyridin-4-yl-[1,2,4]-oxadiazol-5-yl, 3-pyridin-2-yl-[1,2,4]-oxadiazol-5-yl, 5-ethyl-[1,2,4]-oxadiazol-3-yl, 5-phenyl-[1,2,4]-oxadiazol-3-yl, 5-thien-3-yl-[1,2,4]-oxadiazol-3-yl, 5-trifluoromethyl-[1,2,4]-oxadiazol-3-yl, 5-pyridin-4-yl-[1,2,4]-oxadiazol-3-yl, or 5-phenyloxazol-2-yl.

6. The compound of any of the claims 1-5 wherein:
R^{1a} is alkyl, cycloalkyl, aralkyl, heteroaralkyl, cycloalkylalkyl, heterocyclylalkyl, or -alkylene-X⁶—R³² (wherein X⁶ is —NR³³—, —O—, —S(O)ₙ₄—, —CO—, —COO—, —OCO—, —NR³³CO—, —CONR³³—, —NR³³SO₂—, —SO₂NR³³—, —NR³³COO—, —OCONR³³—, —NR³³CONR³⁴, or —NR³³SO₂NR³⁴— (where R³³ and R³⁴ are independently hydrogen, alkyl, or acyl, n4 is 0-2, and R³² is hydrogen, alkyl, haloalkyl, cycloalkyl, cycloalkylalkyl, heterocyclyl, aryl, aralkyl, heteroaryl, heteroaralkyl, heterocyclyl, or heterocyclylalkyl) wherein said alkylene chain in -alkylene-X⁶—R³² is optionally substituted with one to six halo and wherein the aromatic or alicyclic ring in R^{1a} is optionally substituted with one, two, or three R^{e} independently selected from alkyl, haloalkyl, alkoxy, hydroxy, haloalkoxy, halo, nitro, cyano, carboxy, alkoxycarbonyl, aryl, heteroaryl, cycloalkyl, cycloalkylalkyl, aralkyl, heteroaralkyl, amino, monosubstituted amino, disubstituted amino, or acyl; and
R¹ is hydrogen.

7. The compound of any of the claims 1-6 wherein:
R^{1a} is 4,4-dimethylcyclohexylmethyl, 4-ethyl-4-methylcyclohexylmethyl, 4,4-diethylcyclohexylmethyl, 3,3-dimethylcyclohexylmethyl, 3,5-dimethylcyclohexylmethyl, cycloheptylmethyl, cyclooctylmethyl, 2-cyclohexylethyl, 2-cyclohexyl-2-methylpropyl, 2-(1-methylcyclohexyl)ethyl, 2-(1-methylcyclopropyl)ethyl, 2-(1-methylcyclopropyl)-2-methylpropyl, 2-cyclopentylethyl, 2-cyclopentyl-2-methylpropyl, 4-isopropyl-4-methylcyclohexylmethyl, 2-methylcyclohexylmethyl, 4-methoxycyclohexylmethyl, 1-methylcyclopentylmethyl, cyclohexyl, cyclohexylmethyl, 1,4-dimethylcyclopentylmethyl, cyclohexylethyl, cyclohexylmethyl, cyclopentylmethyl1-methylcyclohexylmethyl, 1-methylcyclopentylmethyl, or 1-benzylcyclopropylmethyl; and
R¹ and R² are hydrogen.

8. The compound of any of the claims 1-6 wherein:
R¹⁶ is ethylthiomethyl, ethylsulfinylmethyl, ethylsulfonylmethyl, isopropylthiomethyl, 2-methylthioethyl, 2-methylsulfinylethyl, 2-methysulfonylethyl, 2-methylpropylsulfonylmethyl, isobutylsulfanylmethyl, tert-butylthiomethyl, benzenesulfonylmethyl, 2-phenylsulfanylethyl, 2-phenylsulfonylethyl, naphth-2-ylmethanesulfonylmethyl, biphenyl-2-ylmethanesulfonylmethyl, biphenyl-4-ylmethanesulfonylmethyl, phenylmethanesulfanylmethyl, phenylmethane-sulfinylmethyl, phenylmethanesulfonylmethyl, 2-phenylmethanesulfonylethyl, 4-tert-butylphenylmethanesulfonylmethyl, 2-fluorophenylmethanesulfanylmethyl, 2-fluoro-phenylmethanesulfonylmethyl, 3-fluorophenylmethanesulfonylmethyl, 4-fluorophenylmethanesulfonylmethyl, 2-chlorophenylmethanesulfanylmethyl, 2-chloro-phenylmethanesulfonylmethyl, 3-chlorophenylmethanesulfonylmethyl, 4-chlorophenylmethanesulfonylmethyl, 2-methoxyphenylmethanesulfonylmethyl, 4-methoxyphenylmethanesulfonylmethyl, 2-trifluoromethoxyphenylmethanesulfonylmethyl, 3-trifluoromethoxyphenylmethanesulfonylmethyl, 4-trifluoromethoxyphenylmethanesulfonyl-methyl, 2-trifluoromethylphenylmethanesulfanylmethyl, 2-trifluoromethylphenylmethane-sulfonylmethyl, 3-trifluoromethylphenylmethanesulfonylmethyl, 4-trifluoromethylphenyl-methanesulfonyl-methyl, 2-cyanophenylmethanesulfanylmethyl, 2-cyanophenylmethane-sulfonylmethyl, 3-cyanophenylmethanesulfonylmethyl, 2-bromophenylmethanesulfonylmethyl, 2-nitrophenylmethanesulfanylmethyl, 2-nitrophenylmethanesulfonylmethyl, 2-methylphenyl-methanesulfonylmethyl, 3-methylphenylmethanesulfonylmethyl, 4-methylphenylmethane-sulfonylmethyl, 2-(4-trifluoromethoxy-benzenesulfonyl)ethyl, 2-(3-trifluoromethoxy-benzenesulfonyl)ethyl, 2-(2-trifluoromethoxybenzenesulfonyl)ethyl, 2-difluoromethoxyphenylmethanesulfonylmethyl, 3-difluoromethoxyphenylmethane-sulfonylmethyl, 4-difluoromethoxyphenylmethane-sulfonylmethyl, 2-(4-difluoromethoxy-benzenesulfonyl)ethyl, 2-(2-difluoromethoxybenzenesulfonyl)ethyl, 2-(3-difluoromethoxybenzenesulfonyl)ethyl, 3-chloro-2-fluorophenylmethane-sulfonylmethyl, 3,5-dimethylphenylmethanesulfonylmethyl, 3,5-bis-trifluoromethylphenyl-methanesulfonylmethyl, 2,5-difluorophenylmethanesulfonylmethyl, 2,6-difluorophenylmethanesulfonylmethyl, 2,3-difluorophenylmethane-sulfonylmethyl, 3,4-difluorophenylmethanesulfonylmethyl, 2,4-difluorophenylmethanesulfonylmethyl, 2,5-dichlorophenylmethanesulfonylmethyl, 3,4-dichlorophenylmethanesulfonylmethyl, 2,6-dichlorophenylmethanesulfonylmethyl, 2-fluoro-3-methylphenylmethanesulfonyl-methyl, 4-fluoro-2-trifluoromethoxyphenylmethane-sulfonylmethyl, 2-fluoro-6-trifluoromethylphenylmethanesulfonylmethyl, 2-fluoro-3-trifluoromethylphenyl-methanesulfonylmethyl, 2-fluoro-4-trifluoromethylphenyl-methanesulfonylmethyl, 2-fluoro-5-trifluoromethyl-phenylmethanesulfonylmethyl, 4-fluoro-3-trifluoromethyl-phenylmethanesulfonylmethyl, 2-chloro-5-trifluoromethyl-phenylmethane-sulfonylmethyl, 2,4,6-trifluorophenylmethanesulfonylmethyl, 2,4,5-trifluorophenylmethanesulfonylmethyl, 2,3,4-trifluorophenylmethanesulfonylmethyl, 2,3,5-trifluorophenylmethanesulfonylmethyl, 2,5,6-trifluorophenylmethanesulfonyl-methyl, 3,4,5-trimethoxyphenylmethanesulfonylmethyl, pyridin-2-ylmethanesulfonylmethyl, pyridin-3-ylmethanesulfonylmethyl, pyridin-4-yl-methanesulfonylmethyl, 2-(pyridin-2-ylsulfonyl)ethyl, 2-(pyridin-4-ylsulfonyl)ethyl, oxypyridin-2-ylmethanesulfonylmethyl, cyclohexylmethyl, cyclohexylmethanesulfanylmethyl, cyclohexylsulfinylthiomethyl, cyclohexylmethane-sulfonylmethyl, 2-cyclohexylethanesulfonyl, cyclohexylmethanesulfonylmethyl, cyclopropylmethanesulfonylmethyl, thiophene-2-sulfonylmethyl, 5-chlorothien-2-ylmethane-sulfonylmethyl, or 3,5-dimethyl-isoxazol-4-ylmethanesulfonylmethyl; and
R¹ and R² are hydrogen.

9. The compound of any of the claims 1-6 wherein:
R^{1a} is 2-cyclohexylethyl, cyclohexylmethyl, tert-butylmethyl, 1-methyl-cyclohexylmethyl, 1-methylcyclopentylmethyl, 2,2-difluoro-3-phenylpropyl, 2,2-dichloro-3-phenylpropyl, 1,4-dimethylcyclopentylmethyl, 2,2-dimethyl-3-phenylpropyl, 2-(1,1-difluoro-methoxy)phenylmethane-sulfonylmethyl, 2-(1,1-difluoromethoxy)phenylmethaneoxy-methyl, pyridin-4-ylmethyl, phenylmethanesulfonylmethyl, pyridin-2-ylmethanesulfonylmethyl, pyridin-4-ylmethanesulfonylmethyl, 2-methylpropylsulfonylmethyl, cyclopropylmethanesulfonylmethyl, pyridin-3-ylmethanesulfonylmethyl, 2,6-difluorophenylmethanesulfonylmethyl, 2-pyridin-2-ylsulfonylethyl, 2-phenylsulfonylethyl, benzyloxymethyl, 2,2-dimethylpropyl, cyclopentylmethyl, morpholin-4-ylmethyl, 5-bromothien-2-ylmethyl, pyridin-4-ylmethyl, 2-chlorobenzyl, or 4-fluorobenzyl; and
R¹ and R² are hydrogen.

10. The compound of any of the claims 2-9 wherein:
R³ is methyl, ethyl, isopropyl, cyclopropyl, cyclopentyl, cyclohexyl, phenyl, benzyl, naphthyl, pyrrolidinyl, piperidinyl, morpholinyl, thiomorpholinyl, piperazinyl, furanyl, thienyl, thiazolyl, imidazolyl, pyridinyl, pyrazinyl, or amino where the nitrogen atom is mono or disubstituted with alkyl, and wherein the aromatic or alicylic rings in R³ are optionally substituted with one, two, or three R^{g} independently selected from methyl ethyl, fluoro, chloro, bromo, iodo, hydroxy, oxo, carboxy, cyano, nitro, carboxamide, cyclopropyl, phenyl, pyrrolidinyl, piperidinyl, morpholinyl, thiomorpholinyl, piperazinyl, thienyl imidazolyl, methoxy, acetyl, acetoxy, phenoxy, benzyloxy, methoxycarbonyl, phenoxycarbonyl, benzoyloxy, carbamoyl wherein the nitrogen atom is mono or disubstituted independently with methyl, ethyl or phenyl, acetylamino, benzoylamino, methylthio, phenylthio, phenylsulfonyl, methylsulfonyl, methoxycarbonylamino, phenoxycarbonylamino, methylcarbamoyloxy, phenylcarbamoyloxy, methylsulfonylamino, phenylsulfonylamino, methylaminosulfonyl, phenylaminosulfonyl, amino wherein the nitrogen atom is mono or disubstituted independently with methyl or phenyl; wherein the aromatic or alicyclic rings in R^{g} are further optionally substituted with one, two, or three R^{h} independently selected from methyl, cyclopropyl, phenyl, methoxy, fluoro, chloro, hydroxy, carboxy, or carboxamido.

11. The compound of any of the claims 2-9 wherein:
R³ is phenyl, naphthyl, pyrrolidinyl, piperidinyl, morpholinyl, thiomorpholinyl, furanyl, thienyl, thiazolyl, imidazoly, pyridinyl, or pyrazinyl wherein the aromatic or alicyclic rings in R³ are optionally substituted with one, two, or three R^{g} independently selected from methyl, fluoro, chloro, phenyl, thienyl, methoxy, acetyl, acetoxy, phenoxy, benzyloxy, methoxycarbonyl, carbamoyl wherein the nitrogen atom is mono or disubstituted independently with methyl or phenyl, acetylamino, methylthio, phenylthio, phenylsulfonyl, methylsulfonyl, methoxycarbonylamino, methylcarbamoyloxy, phenylcarbamoyloxy, methylsulfonylamino, phenylsulfonylamino, amino wherein the nitrogen atom is mono or disubstituted independently with methyl or phenyl.

12. The compound of any of the claims 2-9 wherein:
R³ is phenyl, 4-methoxyphenyl, 3-phenoxyphenyl, 4-chlorophenyl, 4-fluorophenyl, 2-fluorophenyl, 2-fluoro-4-chlorophenyl, naphthyl, piperidin-4-yl, morpholin-4-yl, furanyl, thienyl, pyridin-4-yl, or pyrazinyl.

13. The compound of any of the claims 10-12 wherein:
R⁴ is difluoromethyl or trifluoromethyl; and
R^{4′} is hydrogen.

15. The compound of any of the claims 1-9 wherein:
R³ and R^{4′} together with the carbon to which they are attached form cycloalkylene.

16. The compound of any of the claims 1-9 wherein:
R³ and R^{4′} together with the carbon to which they are attached form cyclopentylene, cyclopent-1-enylene, cyclohexylene, or cyclohex-1-enylene.

17. The compound of any of the claims 1-9 wherein:
R³ and R^{4′} together with the carbon to which they are attached from heterocyclylalkylene.

18. A pharmaceutical composition comprising a compound of any of the claims 1-17, individual stereoisomers or mixture of thereof, or a pharmaceutically acceptable salt thereof, in admixture with one or more suitable excipients.

19. A method for treating a disease in an animal mediated by Cathepsin S which method comprises administering to the animal a pharmaceutical composition comprising a compound of any of the claims 1-17, individual stereoisomers or mixture of thereof, or a pharmaceutically acceptable salt thereof, in admixture with one or more suitable excipients.

20. The method of claim 19 wherein the disease is psoriasis.

21. A method of treating a patient undergoing a therapy wherein the therapy causes an immune response in the patient comprising administering to the patient a pharmaceutical composition comprising a compound of claim 1 in admixture with one or more suitable excipients.
